SEQUENCE LISTING (2 sequences)

SEQ ID NO: 1 - protein, 65 aa, Homo sapiens
SHLVKCAEKEKTFCVNGGECFMVKDLSNPSRYLCKCPNEFTGDRCQNYVMASFYKHLGIEFMEAE

SEQ ID NO: 2 - protein, 227 aa, Homo sapiens
SGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQK

(19) United States
(12) Patent Application Publication
(10) Pub. No.: US 2017/0333529 A1
(43) Pub. Date: Nov. 23, 2017

(54) NEUREGULIN IN THE TREATMENT OF FIBROTIC DISORDERS

(71) Applicant: UNIVERSITEIT ANTWERPEN, Antwerpen (BE)

(72) Inventors: Zarha VERMEULEN, Stekene (BE); Vincent SEGERS, Hasselt (BE); Gilles DE KEULENAER, Boechout (BE)

(21) Appl. No.: 15/532,485

(22) PCT Filed: Jan. 20, 2016
(86) PCT No.: PCT/EP2016/051062

§ 371 Date: Jun. 1, 2017

(30) Foreign Application Priority Data
Jan. 20, 2015 (EP) EP15151799.2

(51) Int. Cl. A61K 38/18; A61K 48/00

(52) U.S. Cl. CPC A61K 38/1883; A61K 48/00

(57) ABSTRACT

The present invention relates to the treatment of fibrotic disorders. More particularly, the present invention relates to the use of a neuregulin protein in a method treating, preventing and/or delaying fibrotic skin disorders, fibrotic lung disorders or liver cirrhosis.

### FIELD OF THE INVENTION

The present application provides methods and compositions for treating fibrotic disorders. More particularly, the present invention relates to the use of a neuregulin protein in methods and compositions for treating fibrotic skin disorders, fibrotic lung disorders or liver cirrhosis.

### BACKGROUND OF THE INVENTION

Repair of injured tissue is a fundamental biological process which allows the replacement of injured cells, but when there is an imbalance in the synthesis versus catabolism of the extracellular matrix, the healing process becomes pathogenic and permanent fibrosis can be formed. Fibrosis is the deposition of excess fibrous connective tissue in an organ or tissue. Fibrosis can affect many organs or tissues within the body, such as the lungs, the liver, the heart, the skin, etc. as a result of various events such as infections, mechanical injury, allergic responses and autoimmune reactions. The mechanisms causing pathogenic fibrosis are in many cases unclear and will differ depending on the condition

Fibrotic disorders of the skin, lung and liver, such as idiopathic pulmonary fibrosis, liver cirrhosis, and systemic sclerosis, have an enormous impact on human health, and are a leading cause of morbidity and mortality. Despite this, current therapies, including the administration of immunosuppressive agents such as corticosteroids, are relatively ineffective. Therefore, effective anti-fibrotic therapies for these conditions are urgently needed.

Neuregulin has been shown to enhance repair of the heart after heart failure in mice (Bersell K et al. Cell 2009) and clinical trials for this indication are underway in the US. More recently it was shown that in this context neuregulin also exerts an anti-fibrotic effect in the heart. However, there is no suggestion or motivation to consider whether neuregulin would also affect other fibrotic tissues. Indeed, it is well established that fibroblasts in different tissues are not a homogenous population but differ in their proliferation rate, collagen synthesis as well as MMP expression under basal or inflammatory conditions (Lindner et al., 2012).

### SUMMARY OF THE INVENTION

The present invention is based, at least in part, on the surprising finding that neuregulin has an effect on the pathological fibrosis of different tissues. In particular, as shown in the examples, it has been found that neuregulin-1 (NRG-1) has anti-fibrotic effects on dermal, pulmonary and liver fibrosis.

The present invention is in particular captured by any one or any combination of one or more of the below numbered aspects and embodiments (i) to (xv).

(i) A neuregulin (NRG) protein for use in a method of treating, preventing and/or delaying a fibrotic skin disorder, a fibrotic lung disorder or liver cirrhosis in a mammal.

(ii) The NRG protein for use according to (i), wherein said fibrotic skin disorder is selected from the group consisting of sclerosis, preferably systemic sclerosis (or scleroderma), and hypertrophic scarring.

(iii) The NRG protein for use according to (i), wherein said fibrotic lung disorder is idiopathic pulmonary fibrosis (IPF).

(iv) The NRG protein for use according to any of (i) to (iii), wherein said NRG protein reduces or prevents dermal fibrosis, pulmonary fibrosis, or liver fibrosis.

(v) The NRG protein for use according to any of (i) to (iv), wherein said NRG protein suppresses collagen synthesis and/or fibroblast specific protein-1 (FSP-1) synthesis.

(vi) The NRG protein for use according to any of (i) to (iv), wherein said NRG protein activates the ERK1/2 and/or Akt signalling pathways.

(vii) The NRG protein for use according to any of (i) to (vi), wherein said NRG protein is a neuregulin-1 (NRG-1) protein, a neuregulin-2 (NRG-2) protein, a neuregulin-3 (NRG-3) protein, a neuregulin-4 (NRG-4) protein, or any mixture thereof, preferably a NRG-1 protein.

(viii) The NRG protein for use according to (vii), wherein said NRG protein is a type 1 NRG-1 protein.

(ix) The NRG protein for use according to (viii), wherein said NRG protein is the beta1 isoform of type 1 NRG-1 protein.

(x) The NRG protein for use according to any of (vii) to (ix), wherein said NRG protein comprises an EGF-like domain

(xi) The NRG protein for use according to any of (i) to (x), wherein said NRG protein is to be administered daily.

(xii) The NRG protein for use according to any of (i) to (xi), wherein said NRG protein is to be administered in a daily dose ranging from 0.01 to 100 μg/kg body weight.

(xiii) The NRG protein for use according to any of (i) to (xii), wherein said mammal is a human.

(xiv) A nucleic acid encoding the NRG protein according to any of (i) to (xiii) for use in treating, preventing and/or delaying a fibrotic skin disorder, a fibrotic lung disorder or liver cirrhosis in a mammal.

(xv) A pharmaceutical composition comprising the NRG protein according to any of (i) to (xiii) or the nucleic acid according to (xiv) in an effective amount for use in treating, preventing and/or delaying a fibrotic skin disorder, a fibrotic lung disorder or liver cirrhosis in a mammal.

The appended claims are also explicitly included in the description.

### BRIEF DESCRIPTION OF THE FIGURES

The following description will be illustrated by way of the enclosed figures which provide an illustration of embodiments of the invention only and the invention should in no way be construed to be limited thereto.

FIG. 1: Histological examination of the anti-fibrotic effects of NRG-1 in bleomycin-induced dermal fibrosis. Bleomycin (BLEO) or vehicle (PBS) (CTR) was subcutaneously injected in defined areas of the upper back, and the mice were either treated with rhNRG-1β (NRG) or vehicle (PBS). The injected sections of the skin were stained with Masson's trichrome or Sirius Red. Dermal thickness was determined by measuring the distance between the epidermal-dermal junction and the dermal subcutaneous fat junction. *P<0.05, **P<0.01, *** P<0.005 vs control

FIG. 2: Mean collagen type I (COL1A1), collagen type III (COL3A1) and fibroblast specific protein-1 (FSP-1) mRNA expression levels in skin tissue of vehicle- (CTR), NRG-1 (NRG)-, bleomycin-(BLEO), and bleomycin with NRG-1 (BLEO+NRGsub) treated C57Bl/6 mice. *P<0.05, **P<0.01, *** P<0.005 vs control

FIG. 3: NRG-1 attenuates bleomycin-induced dermal and pulmonary fibrosis and reduces mortality due to pulmonary fibrosis. (A) Bleomycin or vehicle (untreated) was subcutaneously injected in defined areas of the upper back, and the mice were either treated with rhNRG-1β (NRG-1) or vehicle (PBS). The injected sections of the skin were stained with Masson's trichrome or Sirius Red. Dermal thickness was determined by measuring the distance between the epidermal-dermal junction and the dermal subcutaneous fat junction. (B) Mean collagen type I (COL1A1), III (COL3A1), fibronectin-1 and FSP-1 mRNA expression levels of skin tissue of vehicle- (untreated) (PBS), rhNRG-1β-(NRG-1), bleomycin, or bleomycin with rhNRG-1β-(NRG-1) treated C57Bl/6 mice. (C) Western blot analysis of skin tissue of C57Bl/6 mice treated with vehicle-(untreated)(PBS), rhNRG-1β-(NRG-1), bleomycin, or bleomycin with rhNRG-1β-(NRG-1). Collagen type I (COL1A1) and GAPDH bands were quantified by densitometry. (D) Histological examination of the anti-fibrotic effects of NRG-1 in bleomycin-induced pulmonary fibrosis. Bleomycin or vehicle (untreated) was subcutaneously injected in defined areas of the upper back, and the mice were either treated with rhNRG-1β (NRG-1) or vehicle (PBS). Lung fibrosis was analyzed via light microscopy by measuring the area (μm²) of fibrosis normalized to the length of the visceral pleura (μm). (E) Lung weight normalized by tibia length of vehicle- (PBS), rhNRG-1β-(NRG-1), bleomycin, or bleomycin with rhNRG-1β-(NRG-1) treated C57Bl/6 mice. (F) Mean collagen type I (COL1A1), III (COL3A1), fibronectin-1 and FSP-1 mRNA expression levels of lung tissue of vehicle- (PBS), rhNRG-1β-(NRG-1), bleomycin, or bleomycin with rhNRG-1β-(NRG-1) treated C57Bl/6 mice (G) % Survival rate and weight loss of C57Bl/6 mice treated with Bleomycin-(BLEO) or Bleomycin with rhNRG-1β-(NRG-1) (BLEO+NRG-1) *P<0.05, **P<0.01, *** P<0.005 vs control

FIG. 4: NRG-1 attenuates bleomycin-induced dermal and pulmonary fibrosis. (A) Mean metalloproteinase 2 (MMP2), 9 (MMP9), tissue inhibitor of metalloproteinase 1 (TIMP1) and Endothelin-1 mRNA expression levels of fibrotic tissue of vehicle-(untreated) (PBS), rhNRG-1β-(NRG-1), bleomycin, or bleomycin with rhNRG-1β-(NRG-1) treated C57Bl/6 mice (B) Bleomycin or vehicle (untreated) was subcutaneously injected in defined areas of the upper back, and the mice were either treated with rhNRG-1β (NRG-1) or vehicle (PBS). Percentage of alphaSMA-stained dermis.

FIG. 5: Histological examination of the anti-fibrotic effects of NRG-1 in bleomycin-induced pulmonary fibrosis. Bleomycin (BLEO) or vehicle (PBS) (CTR) was subcutaneously injected in defined areas of the upper back, and the mice were either treated with rhNRG-1β (NRG) or vehicle (PBS). Sections of the left lung of each group were stained with Sirius red. Lung fibrosis was analyzed via light microscopy by measuring the area (μm²) of fibrosis normalized to the length of the visceral pleura (μm). *P<0.05, **P<0.01, *** P<0.005 vs control FIG. 6: Lung weight normalized by tibia length of vehicle- (CTR), NRG-1 (NRG)-, bleomycin-(BLEO), and bleomycin with NRG-1 (BLEO+NRGip) treated C57Bl/6 mice. *P<0.05, **P<0.01, *** P<0.005 vs control

FIG. 7: Mean collagen type I (COL1A1), collagen type III (COL3A1) and fibroblast specific protein-1 (FSP-1) mRNA expression levels in lung tissue of vehicle- (CTR), NRG-1 (NRG)-, bleomycin-(BLEO), and bleomycin with NRG-1 (BLEO+NRGip) treated C57Bl/6 mice. *P<0.05, **P<0.01, *** P<0.005 vs control

FIG. 8: Presence of NRG-1 specific receptors in skin and lung fibroblasts. ErbB2, ErbB3 and ErbB4 receptors are present and can be activated by NRG-1 in skin (A) and lung (B) fibroblasts.

FIG. 9: Phosphorylation of ERK1/2 and Akt in skin (A) and lung (B) fibroblasts treated with NRG-1.

FIG. 10: Inhibition of collagen synthesis by NRG-1 in skin and lung fibroblasts. Mean collagen type I (COL1A1, COL1A2) and III (COL3A1) mRNA expression levels of skin (A) and lung (B) fibroblasts stimulated with or without NRG-1. *P<0.05, **P<0.01, *** P<0.005 vs control

FIG. 11: NRG-1 inhibits pro-fibrotic responses of primary fibroblasts in vitro (A) Presence of NRG-1 specific receptors in skin and, lung. ErbB2, ErbB3 and ErbB4 receptors are present and can be activated by rhNRG-1β-(NRG-1) in skin and lung fibroblasts. (B) Treatment of fibroblasts with rhNRG-1β-(NRG-1) attenuated stress-induced upregulation of COL1A1 and COL3A1 mRNA expression levels. (C) NRG-1 induced proliferation of fibroblasts. MMT absorbance in cells treated with vehicle-(PBS), rhNRG-1β-(NRG-1), TGFβ, TGFβ with rhNRG-1β-(TGFβ+NRG1), 5% FBS or 5% FBS with rhNRG-1β-(5% FBS+NRG1) (D-E) Western blot analysis of fibroblasts treated with vehicle-(PBS), rhNRG-1β-(NRG1), TGFβ, TGFβ with rhNRG-1β-(−(TGFβ+NRG1). Alpha-SMA, phosphoSMAD3, GAPDH and total SMAD3 bands were quantified by densitometry. *P<0.05, **P<0.01, *** P<0.005 vs control

FIG. 12: Western blot analysis on cultured fibroblasts treated with NRG-1 for phosphorylated PI3K, total PI3K, phosphorylated STAT3, total STAT, phosphorylated AKT, total AKT, phosphorylated ERK and total ERK.

FIG. 13: Amino acid sequences of a neuregulin fragment (SEQ ID NO: 1) and human neuregulin-1 (SEQ ID NO: 2).

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≧3, ≧4, ≧5, ≧6 or ≧7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); Innis et al., PCR Protocols: A Guide to Methods and Applications, Academic Press: San Diego, 1990. General principles of microbiology are set forth, for example, in Davis, B. D. et al., Microbiology, 3rd edition, Harper & Row, publishers, Philadelphia, Pa. (1980).

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Disclosed herein is a neuregulin (NRG) protein for use in a method of treating, preventing and/or delaying a fibrotic disorder in a subject. In a first aspect, the invention provides a neuregulin (NRG) protein for use in a method of treating, preventing and/or delaying a fibrotic disorder. In particular embodiments, the fibrotic disorder is not a heart fibrotic disorder. In further embodiments, the fibrotic disorder is not a kidney fibrotic disorder. In particular embodiments, the fibrotic disorder is selected from a fibrotic skin disorder, a fibrotic lung disorder or liver cirrhosis.

As used herein, the terms “treating” or “treatment” refer to therapeutic treatment. The terms “treatment”, “treating”, and the like, as used herein also include amelioration or elimination of a developed disease or condition once it has been established or alleviation of the characteristic symptoms of such disease or condition. The terms “preventing” or “prevention” refer to prophylactic measures, wherein the object is to prevent or slow down (lessen) an undesired physiological change or disorder. As used herein these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of the compound or composition of the invention to a patient that is not at the time of administration afflicted with the disease or condition. “Preventing” also encompasses preventing the recurrence or relapse-prevention of a disease or condition or of symptoms associated therewith, for instance after a period of improvement. The terms “delaying” or “delay” may equally refer to postponing the onset of the disease or symptoms, as well as slowing down the progression of the disease or the symptoms.

“Subject” or “patient” are used interchangeably herein and refer to animals, preferably mammals, and specifically include human subjects and non-human mammals. Preferred subjects are humans.

A subject “in need of treatment” includes ones that would benefit from treatment of a given condition, in particular a fibrotic skin disorder, a fibrotic lung disorder or liver cirrhosis. Such subjects may include, without limitation, those that have been diagnosed with said disorder, those prone to contract or develop said disorder and/or those in whom said disorder is to be prevented. Particularly intended are subjects with dermal fibrosis, pulmonary fibrosis, or liver fibrosis, or in whom dermal fibrosis, pulmonary fibrosis, or liver fibrosis, is to be prevented.

The term “fibrosis” generally refers to the development or formation of fibrous connective tissue e.g. as a reparative response to injury or damage. “Fibrosis” refers to the connective tissue deposition that occurs as part of normal healing or to the excess tissue deposition that occurs as a pathological process. As used herein, the term “fibrosis” (also referred to as fibroplasia) particularly refers to the pathological process of excess formation of fibrous connective tissue in a tissue. When fibrosis occurs in response to injury, the term “scarring” can be used as synonym. Fibrosis may occur in many tissues of the body, including lungs, skin, liver, kidney etc.

Fibroblasts are largely responsible for the production and deposition of collagen in skin and lung tissue. When interstitial fibroblasts are activated, e.g. following injury and an inflammatory phase, they develop into myofibroblasts, which produce collagen and other extracellular matrix components. Chronic activation of these myofibroblasts promotes excessive accumulation of extracellular matrix which can lead to formation of a permanent fibrotic scar.

The main liver cells that produce matrix are Hepatic Stellate Cells (HSC). This resident cell population exists in a resting phenotype, but on activation they transform to adopt a myofibroblast phenotype capable of secreting collagen and other extracellular matrix components.

The present invention particularly relates to a neuregulin protein for use in a method of treating, preventing and/or delaying a fibrotic skin disorder, a fibrotic lung disorder, or liver cirrhosis.

With “pulmonary fibrosis”, “lung fibrosis” or “fibrotic lung disorder” is meant herein pathological fibrosis in the lungs. Diseases which are primarily characterized by fibrosis in the lung are also referred to as interstitial lung diseases. Pulmonary fibrosis is characterized by a dense distribution of paraseptal and subpleural collagen. Matrix scars are formed in the lung tissues, leading to serious breathing problems. Pulmonary fibrosis can be caused by many conditions, including chronic inflammatory processes (like sarcoidosis or Wegener's granulomatosis), infections, environmental agents (e.g. asbestos, silica, exposure to certain gases), exposure to ionizing radiation (such therapy to treat tumors of the chest), chronic conditions (e.g. lupus, rheumatoid arthritis), and certain drugs, such as nitrofurantoin and methotrexate. In cases where the underlying cause is not clear, the term idiopathic pulmonary fibrosis is used.

Symptoms of pulmonary fibroses are mainly: shortness of breath, particularly with exertion, chronic dry, hacking coughing, fatigue and weakness, chest discomfort including chest pain, and loss of appetite and rapid weight loss.

In preferred embodiments envisaged herein, the fibrotic lung disorder is idiopathic pulmonary fibrosis (IPF). In further embodiments, the hypersensitivity pneumonitis, cryptogenic penumonitis, acute intersititial penumonitis, desquamative interstitial pneumonitis, sarcoidosis or asbestosis.

With “dermal fibrosis” or “skin fibrosis” is meant herein pathological fibrosis in skin tissue. Dermal fibrosis is characterized by thick and rigid skin caused by excessive accumulation of extracellular matrix proteins.

“Fibrotic skin disorders” or “fibrotic dermal disorders” are cutaneous disorders characterized by excessive scarring of the skin due to pathologic skin fibrosis. Clinically, skin fibrosis manifests as thickened, tightened, and hardened areas of skin. Ultimately, skin fibrosis may lead to dermal contractures that affect the ability to flex and extend the joints. Non-limiting examples of fibrotic skin disorders include scleroderma in both, localized (morphea, linear scleroderma) and systemic form (scleroderma), hypertrophic scarring, keloids, mixed connective tissue disease, scleredema, scleromyxedema, eosinophilic fasciitis. In preferred embodiments, the fibrotic skin disorder is selected from the group consisting of hypertrophic scarring, and sclerosis, in particular systemic sclerosis (or scleroderma).

With “liver fibrosis” is meant herein pathological fibrosis in liver tissue. Underlying causes of liver fibrosis may be alcoholism, fatty liver disease, and hepatitis infection.

As used herein, “fibrotic disorder” or “fibroproliferative disorder” refers to a pathological condition due to the formation of excess fibrous connective tissue. Non-limiting examples of fibrotic disorders include pulmonary fibroses (such as idiopathic pulmonary fibrosis, chronic fibrosis (or mucoviscidosis)) fibrotic skin disorders (such as systemic sclerosis or scleroderma, and hyperthrophic scarring), liver cirrhosis, progressive kidney disease, cardiovascular disease, and macular degeneration. Pathological fibrosis in the respective tissues or organs is a common hallmark of these disorders.

“Liver cirrhosis” is a slowly progressing disease in which healthy liver tissue is replaced with scar tissue, thereby preventing the liver from functioning properly. The scar tissue blocks the flow of blood through the liver and thereby slows the processing of nutrients, hormones, drugs, and naturally produced toxins. It also slows the production of proteins and other substances made by the liver. Liver cirrhosis may cause a wide range of symptoms, including tendency to bleed or bruise early, fatigue, jaundice or yellowing of the skin and eyes, ascites or fluid build-up in the abdomen, weight loss, itchy skin, nausea, swelling in the legs, disorientation and drowsiness, slurred speech and development of spider-like vessels underneath the skin surface.

The present inventors have found that administration of a neuregulin protein to a mammal reduces or prevents fibrosis in different tissues where this has not previously been demonstrated, in particular dermal fibrosis, pulmonary fibrosis or liver fibrosis. The reduction or prevention of dermal fibrosis by neuregulin can be established by a reduction or prevention of the up-regulation of dermal fibrotic markers such as collagen type I, collagen type III, and fibroblast specific protein-1 (FSP-1). The reduction or prevention of pulmonary fibrosis can be established by a reduction or prevention of the up-regulation of pulmonary fibrotic markers such as collagen type I, collagen type III, and fibroblast specific protein-1 (FSP-1). Accordingly, in particular embodiments, methods and compositions are provided whereby administration of a neuregulin protein to a mammal suppresses or prevents collagen synthesis, preferably collagen I and/or collagen II synthesis, and/or FSP-1 synthesis. The reduction or prevention of liver fibrosis can be established by a reduction or prevention of the up-regulation of liver fibrotic markers such as collagen type I, collagen type III, and fibroblast specific protein-1 (FSP-1). As used herein, “synthesis” refers to protein expression. Suppression or prevention of protein synthesis may relate to suppression or prevention of transcription of the protein encoding gene and/or suppression or prevention of translation of the protein encoding mRNA, both of which can be assayed by routine techniques, such as respectively Western blot or Q-PCR. In a preferred embodiment, suppression or prevention of collagen synthesis, preferably collagen type I and/or collagen type III synthesis, and/or FSP-1 synthesis relates to a suppression or prevention of transcription of the respective genes. Suppression or prevention of one or both of these genes preferably occurs in fibroblasts, more preferably skin fibroblasts or lung fibroblasts, or hepatic stellate cells. Accordingly, in particular embodiments, the neuregulin protein suppresses or prevents collagen, preferably collagen I and/or collagen III, and/or FSP-1 synthesis in fibroblasts, more particularly skin fibroblasts or lung fibroblasts, in hepatic stellate cells.

Without being bound by theory, it is believed that the neuregulin protein activates various signaling pathways, such as the Akt signal transduction pathway and the Erk signal transduction pathway, so as to ensure its anti-fibrotic as observed herein. Accordingly, in particular embodiments, the invention relates to a neuregulin protein for use in the treatment, prevention and/or delay of a fibrotic disorder, in particular a fibrotic skin disorder, a fibrotic lung disorder or liver cirrhosis, wherein said neuregulin protein activates the Akt and/or Erk signaling pathways. Methods for identifying activation of signal transduction pathways are well known in the art. The Akt and Erk signaling pathways are also well known in the art. Accordingly, the skilled person is amply capable of evaluating the activation of either one of these pathways. By means of further guidance, activation of these pathways may for instance be determined by measurement of phosphorylated Akt, respectively Erk. In this context, phosphorylation (or increased phosphorylation) of Akt or Erk indicates activation (or increased activation) of respectively the Akt and Erk pathways. Activation of one or both of these pathways preferably occurs in fibroblasts, more preferably skin fibroblasts or lung fibroblasts, or hepatic stellate cells.

In particular embodiments, the invention relates to a neuregulin protein for use in the treatment, prevention and/or delay of a fibrotic disorder, in particular a fibrotic skin disorder, a fibrotic lung disorder or liver cirrhosis, wherein said neuregulin protein suppresses phosphoinositide 3 kinase (PI3K) and STAT3. Inhibition of these pathways may for instance be determined by measurement of phosphorylated PI3K and STAT3. In this context, no phosphorylation (or decreased phosphorylation) of PI3K or STAT3 indicates inhibition (or decreased activation) of respectively the PI3K and STAT3 signaling pathways. Activation of one or both of these pathways preferably occurs in fibroblasts, more preferably skin fibroblasts or lung fibroblasts, or hepatic stellate cells.

As used herein, the term “neuregulin protein” refers to a protein of the neuregulin family. Neuregulins or neuroregulins are a family of four structurally related proteins that are part of the EGF family of proteins. The neuregulin family includes: (1) neuregulin-1 (NRG-1), with isoforms stemming from alternative splicing: type I NRG-1; alternative names: Heregulin, NEU differentiation factor (NDF), or acetylcholine receptor inducing activity (ARIA); type II NRG-1; alternative name: Glial Growth Factor-2 (GGF2); type III NRG-1; alternative name: Sensory and motor neuron-derived factor (SMDF); type IV NRG-1; type V NRG-1; type VI NRG-1; (2) Neuregulin-2 (NRG-2); (3) Neuregulin-3 (NRG-3); (4) Neuregulin-4 (NRG-4).

In a preferred embodiment, the neuregulin protein as used herein is NRG-1. In a more preferred embodiment, the neuregulin protein as used herein is type I NRG-1 (heregulin). In an even more preferred embodiment, the neuregulin protein as used herein is the beta isoform of NRG-1, preferably NRG-1 type I, i.e. NRG-1 type I β. In a further preferred embodiment, the neuregulin protein as used herein is the beta1 isoform of NRG-1, preferably NRG-1 type I, i.e. NRG-1 type I β1.

In certain embodiments, the neuregulin protein as referred to herein may be either one or a mixture of two or more of the above recited family members.

The neuregulin protein as taught herein may be used in monomeric form or in multimeric or multivalent form, preferably in dimeric or bivalent form. Dimers of a neuregulin protein are not known to be naturally occurring and, as a result, are referred to herein as being synthetic or engineered. In certain embodiments, the neuregulin protein is used in dimeric form. Neuregulin multimers or dimers as described herein comprise a neuregulin protein in monomeric form and one or more of the same or another ErbB2, ErbB3 or ErbB4 ligand. The monomers of the neuregulin dimer may be identical (i.e. neuregulin homodimer) or different (i.e. neuregulin heterodimer). Accordingly, contemplated herein are the following non-limiting examples of neuregulin dimers: NRG2b-NRG2b, NRG2b-NRG2a, NRG2b-NRG1B3, NRG2b-NRG1α, NRG2b-NRG1B, NRG2b-NRG2, NRG2b-NRG3, NRG2b-NRG4, NRG2a-NRG2α, NRG2a-NRG1B3, NRG2a-NRG1α, NRG2a-NRG1B, NRG2a-NRG2, NRG2a-NRG3, NRG2a-NRG4, NRG1B3-NRG1B3, NRG1B3-NRG1α, NRG1B3-NRG1B, NRG1B3-NRG2, NRG1B3-NRG3, NRG1B3-NRG4, NRG1a-NRG1α, NRG1a-NRG1B, NRG1a-NRG2, NRG1a-NRG3, NRG1a-NRG4, NRG1B-NRG1B, NRG1B-NRG2, NRG1B-NRG3, NRG1B-NRG4, NRG2-NRG2, NRG2-NRG3, NRG2-NRG4, NRG3-NRG3, NRG3-NRG4, and NRG4-NRG4. The neuregulin monomers are typically linked with a linker in the neuregulin dimers described herein. The linker may comprise a coiled coil, a peptide spacer, a water soluble flexible polymer (such as e.g. polyethylene oxide, dextran, polyacrylic acid and polyacrylamide), or a combination thereof. The neuregulin dimers can be produced with e.g. the methods described in paragraphs 104 to 107 of US application US 2013/0196911, which is specifically incorporated by reference herein, or methods otherwise described in the art. Methods for producing ligand dimers such as neuregulin dimers are known in the art and described in e.g. PCT application WO2010033249, which is specifically incorporated by reference herein.

It is to be understood that the neuregulin protein as referred to herein is preferably the mature neuregulin protein (i.e. the cleaved pro-neuregulin protein, which contains the EGF-like domain), which may or may not contain a signal peptide, but preferably does not contain a signal peptide. The neuregulin protein as referred to herein may be a naturally occurring neuregulin protein, for instance which is isolated from a specific host. Alternatively, the neuregulin protein as referred to herein may be recombinantly produced (e.g. in E. coli, yeast, CHO cell lines, or other hosts).

In particular embodiments, the neuregulin protein as referred to herein comprises or consists of a human neuregulin protein. In a preferred embodiment, the neuregulin protein as used herein is human NRG-1. In a more preferred embodiment, the neuregulin protein as used herein is type I human NRG-1 (heregulin). In an even more preferred embodiment, the neuregulin protein as used herein is the beta isoform of human NRG-1, preferably human NRG-1 type I, i.e. human NRG-1 type I β. In a further preferred embodiment, the neuregulin protein as used herein is the beta1 isoform of human NRG-1, preferably human NRG-1 type I, i.e. human NRG-1 type I β1.

In particular embodiments, the neuregulin protein as referred to herein also encompasses a homologue, an orthologue, or a functional fragment or variant of a neuregulin protein, such as a human neuregulin protein. The terms “orthologue”, “homologue”, “functional variant”, and “functional fragment” are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. A “functional variant” or “functional fragment” of a protein as used herein refers to a variant or fragment of such protein which retains at least partially the activity of that protein. Functional variants or fragments may include mutants (which may be insertion, deletion, or replacement mutants), including polymorphs, etc. Functional variants or fragments may be naturally occurring or may be man-made.

In particular embodiments, the homologue, orthologue, functional variant, or functional fragment of the neuregulin protein as referred to herein has a sequence identity of at least 80%, more preferably at least 85%, even more preferably at least 90%, such as for instance at least 95% with one or more of the human neuregulin proteins. It is to be understood that when referring to sequence alignments, the sequence identity is to be determined based on the shortest sequence to be aligned. For instance, sequence alignment of a neuregulin fragment which is shorter than the neuregulin full length protein is to be determined based on the length of the fragment. In a preferred embodiment, the homologue, orthologue, functional variant, or functional fragment of the neuregulin protein as referred to herein has a sequence identity of at least 80%, more preferably at least 85%, even more preferably at least 90%, such as for instance at least 95% with human NRG-1. In a more preferred embodiment, the homologue, orthologue, functional variant, or functional fragment of the neuregulin protein as referred to herein has a sequence identity of at least 80%, more preferably at least 85%, even more preferably at least 90%, such as for instance at least 95% with type I human NRG-1 (heregulin). In an even more preferred embodiment, the homologue, orthologue, functional variant, or functional fragment of the neuregulin protein as referred to herein has a sequence identity of at least 80%, more preferably at least 85%, even more preferably at least 90%, such as for instance at least 95% with the beta isoform of human NRG-1, preferably human NRG-1 type I, i.e. human NRG-1 type I β. In a further preferred embodiment, the homologue, orthologue, functional variant, or functional fragment of the neuregulin protein as referred to herein has a sequence identity of at least 80%, more preferably at least 85%, even more preferably at least 90%, such as for instance at least 95% with the beta1 isoform of human NRG-1, preferably human NRG-1 type I, i.e. human NRG-1 type I β1. In a further preferred embodiment, the homologue, orthologue, functional variant, or functional fragment of the neuregulin protein as referred to herein has a sequence identity of at least 80%, more preferably at least 85%, even more preferably at least 90%, such as for instance at least 95%, more particularly is 100% identical to SEQ ID NO:2.

In particular embodiments, the neuregulin protein, functional fragment, functional variant, orthologue, or homologue as referred to herein, such as a human neuregulin protein, functional fragment, functional variant, orthologue, or homologue comprises, consists essentially of, or consists of an EGF-like domain. EGF-like domains are well known in the art and can be easily identified by routine techniques involving sequence alignments. A protein BLAST analysis also outputs conserved domains, such that the presence of an EGF-like domain can be readily evaluated. The EGF-like domains of all neuregulins have for instance also been annotated in protein and nucleic acid databases, which can for instance be accessed at the ncbi website. The skilled person is therefore capable to easily determine if the neuregulin protein, functional fragment, functional variant, orthologue, or homologue as referred to herein comprises an EGF-like domain.

In particular embodiments, the EGF-like domain containing homologue, orthologue, functional variant, or functional fragment of the neuregulin protein as referred to herein has a sequence identity of at least 80%, more preferably at least 85%, even more preferably at least 90%, such as for instance at least 95% with human neuregulin. In a preferred embodiment, the EGF-like domain containing homologue, orthologue, functional variant, or functional fragment of the neuregulin protein as referred to herein has a sequence identity of at least 80%, more preferably at least 85%, even more preferably at least 90%, such as for instance at least 95% with human NRG-1. In a more preferred embodiment, the EGF-like domain containing homologue, orthologue, functional variant, or functional fragment of the neuregulin protein as referred to herein has a sequence identity of at least 80%, more preferably at least 85%, even more preferably at least 90%, such as for instance at least 95% with type I human NRG-1 (heregulin). In an even more preferred embodiment, the EGF-like domain containing homologue, orthologue, functional variant, or functional fragment of the neuregulin protein as referred to herein has a sequence identity of at least 80%, more preferably at least 85%, even more preferably at least 90%, such as for instance at least 95% with the beta isoform of human NRG-1, preferably human NRG-1 type I, i.e. human NRG-1 type I β. In a further preferred embodiment, the EGF-like domain containing functional fragment of the neuregulin protein as referred to herein has a sequence identity of at least 80%, more preferably at least 85%, even more preferably at least 90%, such as for instance at least 95% with the beta1 isoform of human NRG-1, preferably human NRG-1 type I, i.e. human NRG-1 type I β1. In the context of the present invention, a functional fragment, refers to a fragment of a neuregulin protein which can bind to and activate a cognate ErbB receptor. Similarly, a functional variant or a homologue, refers to a molecule which can bind to and activate a cognate ErbB receptor. In a particular embodiment, the functional fragment of the neuregulin protein as referred to herein corresponds to the sequence of the EGF domain of human neuregulin-1 or Heregulin-β1, i.e. which corresponds to the N-terminal fragment of NRG-1. In a preferred embodiment, the functional fragment of the neuregulin protein as referred to herein has a sequence identity of at least 80%, more preferably at least 85%, even more preferably at least 90%, such as for instance at least 95%, more particularly 100% sequence identity with the corresponding part of the sequence of SEQ ID NO: 1. Examples of functional variants of a neuregulin protein are provided in US2014031284, WO03/099300, U.S. Pat. No. 537,060 and U.S. Pat. No. 6,136,558.

Methods for comparing sequences and determining sequence identity are well known in the art. By means of example, percentage of sequence identity refers to a percentage of identical nucleic acids or amino acids between two sequences after alignment of these sequences. Alignments and percentages of identity can be performed and calculated with various different programs and algorithms known in the art. Preferred alignment algorithms include BLAST (Altschul, 1990; available for instance at the NCBI website) and Clustal (reviewed in Chenna, 2003; available for instance at the EBI website). Preferably, BLAST is used to calculate the percentage of identity between two sequences, such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3).

In particular embodiments, the neuregulin protein, or the homologue, orthologue, functional variant, or functional fragment of the neuregulin protein as referred to herein comprises, consists essentially of, or consists of a polypeptide having a sequence as set forth in SEQ ID NO: 1 or SEQ ID NO: 2, or has a sequence identity of at least 80%, more preferably at least 85%, even more preferably at least 90%, such as for instance at least 95% with a polypeptide having a sequence as set forth in SEQ ID NO: 1 or SEQ ID NO: 2.

In particular embodiments, the homologue of the neuregulin protein is a protein or compound capable of binding to and activating the ErbB4 receptor. Examples of proteins and molecules which can be identified based on their ability to bind and activate the ErbB4 receptor are activating antibodies or small molecules. In particular embodiments, these molecules specifically activate the ErbB4 receptor.

A neuregulin protein as described herein, or the homologue, orthologue, functional variant, or functional fragment of the neuregulin protein as referred to herein, optionally together with a pharmaceutically acceptable carrier, may be administered by any suitable mode of application, e.g. i.d., i.v., i.p., i.m., intranasally, orally, subcutaneously, etc. and in any suitable delivery device (O'Hagan et al., Nature Reviews, Drug Discovery 2 (9), (2003), 727-735). The proteins of the present invention are preferably formulated for intravenous, subcutaneous, intradermal or intramuscular administration (see e.g. “Handbook of Pharmaceutical Manufacturing Formulations”, Sar-faraz Niazi, CRC Press Inc, 2004). Accordingly, the present invention also relates to a pharmaceutical composition comprising a neuregulin protein as described herein, or a homologue, orthologue, functional variant, or functional fragment of the neuregulin protein as referred to herein, optionally together with a pharmaceutically acceptable carrier, fur use in treating, preventing, and/or delaying a fibrotic skin disorder, a fibrotic lung disorder or liver cirrhosis in a mammal.

As used herein, “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutically active substances is well known in the art. Such materials should be non-toxic and should not interfere with the activity of the neuregulin proteins.

In an aspect, the invention also relates to a pharmaceutical composition comprising the neuregulin protein, or the homologue, orthologue, functional variant, or functional fragment thereof, as defined herein elsewhere, in an effective amount for use in treating, preventing and/or delaying a fibrotic skin disorder, a fibrotic lung disorder or liver cirrhosis in a mammal.

As used herein, the term “effective amount” refers to the amount or dose of the protein, the nucleic acid, or the composition, such as a pharmaceutical composition, which attains a therapeutic or prophylactic effect in the subject to which it is administered. An effective amount is an amount which can elicit a biological or medicinal response in a tissue, system, subject to which the protein, nucleic acid, or composition is administered, and in particular can prevent or alleviate one or more of the local or systemic symptoms or features of a disease or condition being treated.

In an embodiment, the neuregulin protein, homologue, orthologue, functional variant, or functional fragment thereof, as defined herein elsewhere, is to be administered in a concentration ranging from 0.01 to 100 μg/kg, i.e from 0.01 to 100 μg/kg body weight of the subject it is to be administered to, preferably from 0.05 to 50 μg/kg, more preferably from 0.1 to 10 μg/kg. In another embodiment, the neuregulin protein, homologue, orthologue, functional variant, or functional fragment thereof, as defined herein elsewhere, is to be administered in a concentration ranging from 0.01 to 100 μg/kg/day, i.e from 0.01 to 100 μg/kg body weight of the subject it is to be administered to per day, preferably from 0.05 to 50 μg/kg/day, more preferably from 0.1 to 10 μg/kg/day. In another embodiment, the neuregulin protein, homologue, orthologue, functional variant, or functional fragment thereof, as defined herein elsewhere, is to be administered in a concentration ranging from 0.01 to 100 μg/kg/week, i.e from 0.01 to 100 μg/kg body weight of the subject it is to be administered to per week, preferably from 0.05 to 50 μg/kg/week, more preferably from 0.1 to 10 μg/kg/week.

In an embodiment, the neuregulin protein, homologue, orthologue, functional variant, or functional fragment thereof, as defined herein elsewhere, is to be administered in a concentration ranging from 10 to 1000 pmol/kg, i.e from 10 to 1000 pmol/kg body weight of the subject it is to be administered to, preferably 30 to 500 pmol/kg, more preferably from 50 to 100 pmol/kg. In another embodiment, the neuregulin protein, homologue, orthologue, functional variant, or functional fragment thereof, as defined herein elsewhere, is to be administered in a concentration ranging from 10 to 1000 pmol/kg/day, i.e from 10 to 1000 pmol/kg body weight of the subject it is to be administered to per day, preferably 30 to 500 pmol/kg/day, more preferably from 50 to 100 pmol/kg/day. In another embodiment, the neuregulin protein, homologue, orthologue, functional variant, or functional fragment thereof, as defined herein elsewhere, is to be administered in a concentration ranging from 10 to 1000 pmol/kg/week, i.e from 10 to 1000 pmol/kg body weight of the subject it is to be administered to per week, preferably 30 to 500 pmol/kg/week, more preferably from 50 to 100 pmol/kg/week.

It will be understood by the skilled person that the duration of the treatment may vary, possibly depending on the desired outcome, for instance improvement of one or more symptoms, complete cure, etc. For instance, the neuregulin protein, such as a pharmaceutical composition comprising a neuregulin protein, may be administered only once. Alternatively, the neuregulin protein may be administered on a daily basis for a specified duration, such as for instance during or at least during 2, 3, 4, 5, 6, 7, or more days, which may or may not be consecutive days. The neuregulin protein may also be administered multiple times per day, such as at least 2, 3, 4, 5, 6, 7 or more times per day. The neuregulin protein may for instance also be administered multiple times per week, such as for instance at least 2, 3, 4, or more times per week. The neuregulin protein may for instance also be administered weekly, every 2, 3, 4 or more weeks. The neuregulin protein may for instance also be administered monthly, every 2, 3, 4 or more months.

It will be further understood by the skilled person that the mode of administration of the neuregulin protein may vary. For instance, the neuregulin protein, such as a pharmaceutical composition comprising a neuregulin protein, may be administered in bolus, or may alternatively be administered during a prolonged time frame. For instance, the neuregulin protein may be administered, e.g. as a drip, over a period of several minutes or hours, such as for instance during 5, 6, 7, 8, 9, 10, 11, 12, 13, 14, 15, 16, 17, 18, 19, 20, or more minutes, such as for instance during 10, 20, 30, 40, 50, 60, or more minutes, such as for instance during, 0.5, 1, 1.5, 2, 2.5, 3, 3.5, 4, or more hours, such as for instance during 4, 6, 8, 10, 12, 14, 16, 18, 20, 22, 24, or more hours.

In another aspect, the invention relates to a nucleic acid comprising a nucleic acid sequence encoding a neuregulin protein as described herein, or a homologue, orthologue, functional variant, or functional fragment of the neuregulin protein as referred to herein for use in treating, preventing, and/or delaying a fibrotic skin disorder, a fibrotic lung disorder or liver cirrhosis in a subject. Preferably, said nucleic acid is an eukaryotic expression vector which comprises a nucleic acid sequence encoding a neuregulin protein as described herein, or a homologue, orthologue, functional variant, or functional fragment of the neuregulin protein as referred to herein. Such vectors are well known in the art, and may include regulatory elements and/or tissue specific promoters such that expression of the encoding sequence can be modulated, such as to result in tissue specific expression, but also inducible expression, or combinations thereof.

Additionally the invention relates to methods for treating, preventing and/or delaying a fibrotic disorder, such as a fibrotic skin disorder, a fibrotic lung disorder or liver cirrhosis, as described herein above comprising administering to a subject in need thereof a neuregulin protein, or a homologue, orthologue, functional variant, or functional fragment thereof, as defined herein elsewhere, or a nucleic acid encoding such protein, as defined herein elsewhere. In particular embodiments, the patient in need thereof does not suffer from a heart condition.

Additionally, the invention relates to the use of a neuregulin protein, or a homologue, orthologue, functional variant, or functional fragment thereof, as defined herein elsewhere, or a nucleic acid encoding such protein, as defined herein elsewhere for the preparation of a medicament for treating, preventing and/or delaying fibrotic skin disorder, a fibrotic lung disorder or liver cirrhosis, as defined herein elsewhere.

The aspects and embodiments of the invention are further supported by the following non-limiting examples.

### EXAMPLES

I. Methods and Protocols

Experimental Animals and Study Designs

Animals

C57BL/6 mice, aged 8 weeks and weighting 20 to 25 g, were obtained from The Jackson Laboratory. Mice were maintained under standard laboratory conditions, 12 hours light-dark cycles with access to normal chow and drinking water at libitum. All experiments performed are approved by the ethical committee of animals of the University of Antwerp and conform to the Guide for the Care and Use of Laboratory Animals published by the US National Institutes of Health (NIH Publication No. 85-23, revised 1996).

Study Design

### Experiment A: Induction of Dermal and Lung Fibrosis

Fibrosis was induced in 8 week old female C57BL/6 mice (n=40) by subcutaneous injection of bleomycin. Bleomycin was dissolved in phosphate-buffered saline (PBS; 0.01 M, pH 7.4) at a concentration of 0.5 mg/mL and 100 μL was administered 5 days a week during 4 weeks by subcutaneous injection in defined areas of the upper back as described in Wynn (2007). Bleomycin-treated mice were randomized into treatment groups with (1) rhNRG-1β intraperitoneal injection (20 μg·kg⁻¹·day⁻¹, PeProtech), (2) rhNRG-1β subcutaneous injection (20 μg·kg⁻¹·day⁻¹), or (3) received no treatment (injection with vehicle (PBS)). Control mice were injected with vehicle (PBS) instead of bleomycin and were treated with either (1) rhNRG-1β or (2) vehicle (PBS).

### Experiment B: Induction of Dermal and Lung Fibrosis

Dermal fibrosis was induced in 8-week old C57Bl/6 mice (n=10-12/group) by daily interscapular subcutaneous injections of bleomycin (1 U/kg, 7 days or 4 weeks, Sigma). Pulmonary fibrosis was induced in 12-week old C57Bl/6 mice (n=10/group) by a single intratracheal instillation of bleomycin (4U/kg, 7 days or 2 weeks) using a 27-gauge needle as previously described (Olman et al., 1995). Survival rates were analyzed throughout 25 days after bleomycin installation. Control mice were sham treated with phosphate buffered saline (PBS, Invitrogen). Simultaneously, subgroups were treated with daily intraperitoneal injections of either recombinant human (rh)NRG-1 (20 μg·kg⁻¹·day⁻¹, Peprotech) or PBS.

ErbB^{F/+}; s100a4-Cre knock-out (KO) mice were generated by breeding s100a4-Cre mice (Jackson laboratories) with mice carrying loxP-flanked (F) ErbB4 alleles (MMRRC) (Strutz et al., 1995). The fibrotic models were induced in these mice and ErbB4^{+/+}; s100a4-Cre littermates were used as a control.

Histological Analysis of Dermal and Pulmonary Fibrosis

### Experiment A

Dermal Fibrosis

The injected sections of skin of the upper back were fixed in 4% buffered formalin and embedded in paraffin for histological staining. Five-micrometer sections were stained with Sirius red and Masson's trichrome to determine the dermal thickness. Dermal thickness was analyzed via light microscopy (Olympus U-TU1X-2, Japan) at a 4-fold magnification, by measuring the distance between the epidermal-dermal junction and the dermal subcutaneous fat junction.

Pulmonary Fibrosis

The left lung was taken from each mouse and was fixed in 4% buffered formalin and was embedded in paraffin for histological staining. Longitudinal sections of the lung were stained with Sirius red and Masson's trichrome and evaluated for fibrosis. Lung fibrosis was analyzed via light microscopy (Olympus U-TU1X-2, Japan) at a 4-fold magnification, by measuring the area (μm²) of fibrosis normalized to the length of the visceral pleura (μm).

### Experiment B

The apex of the skin and left lung were fixed in 4% buffered formalin and embedded in paraffin. Sections were stained with Sirius red, Masson's trichrome, DAB according to the manufacturer's instructions or with antibodies specific for αSMA (Abcam), collagen type I (T3313G, Campro Scientific) and Mac3 (PharMingen). Images were acquired with a microscope (Olympus U-TU1X-2) and analyzed with ImageJ software. Pulmonary and cardiac fibrosis was quantified by calculating the percent rate of stained ECM area to total tissue area in digitalized microscopic images. Severity of dermal fibrosis was determined by measuring the dermal thickness between the epidermal-dermal junction and the dermal-hypodermal junction. Inflammation was quantified by calculating the percentage of neutrophil and macrophage infiltration. Quantification was performed by a person blinded to the treatment protocol.

### Experiment A: Lung and Skin Fibroblasts

Primary cultures of skin and lung fibroblasts were established using a method introduced by Takashima (2001). Briefly, a small sample of skin and lung was taken from Sprague-Dawley rats and placed on tissue culture dishes. After a view days, fibroblast outgrowth was seen. Fibroblasts were passaged in Dulbecco's modified Eagle's medium (DMEM) enriched with 10% Fetal bovine serum (FBS) in an incubator at 37° C. with 5% CO₂. Medium was changed every 2 to 3 days. Presence and activation of ErbB receptors in fibroblasts was analyzed using Western Blotting and immunoprecipitation after stimulation with rhNRG-1β for 30 minutes, or not. The effect of rhNRG-1β on collagen mRNA expression was measured using Real-Time PCR.

### Experiment B: Lung and Skin Fibroblasts

Primary fibroblasts were cultured form mouse tissue biopsies of lung and skin as previously described (Sanada et al, 2007 and Takashima et al., 2001). Fibroblasts were cultured in Dulbecco's Modified Eagle's Medium (DMEM, Invitrogen) containing 10% fetal bovine serum (FBS, Invitrogen). Cells were used in passage 2 or 3 and were serum-starved for 24 hours prior to experiments. Primary fibroblasts were exposed to rhNRG-1β (50 ng/mL, PeproTech).

Western Blotting and Immunoprecipitation

### Experiment A

Fibroblasts were collected in lysis buffer consisting of 20 mM Tris, 137 mM NaCl, 10% (vol/vol) glycerol, 1% (vol/vol) Nonidet P-40, and 2 mM ethylenediaminetetraacetic acid and supplemented with protease and phosphatase inhibitors (Complete; Roche and Sigma, respectively). Immunoprecipitation (with specific ErbB antibodies) followed by Western blot analysis (with a phospho-tyrosin antibody) was performed as previously described in Lemmens et al. (2011). In brief, equal amounts of cell lysates were incubated with primary antibody at 4° C. overnight, in which thereafter, protein NG plus agarose beads (Santa Cruz, abcam) were added. Proteins were separated on NuPAGE® BisTris gels (Invitrogen) and electrotransferred to polyvinylidene difluoride membrane (Pierce). Membranes were blocked with 5% BSA and incubated with primary antibodies after which secondary horseradish peroxidase-conjugated antibody was applied. Antibodies used were ErbB2, ErbB3, ErbB4, (phospho-)Akt and (phospho-)ERK1/2 (Santa Cruz, Abcam). The signal was revealed with Supersignal West Pico chemiluminescent substrate (Pierce)

### Experiment B

Western analysis and immunoprecipitation were performed as described previously (Lemmens et al., 2011). Membranes (PVDF; Invitrogen) were incubated overnight with primary antibodies ErbB2, ErbB3, ErbB4, αSMA and GAPDH (all from Santa Cruz), collagen-1 (Abcam) and p-SMAD3, SMAD3, p-p44/42, p44/42, p-Akt, Akt, p-PI3K and PI3K (Cell signaling) at 4° C., all diluted at 1:1,000, detected with HRP-conjugated antibodies (Santa Cruz) and enhanced using chemiluminescence (Invitrogen).

Cell Viability Assay

Cell proliferation was determined using a methyl thiazolyl tetrazolium (MTT) assay (Invitrogen). Briefly, fibroblasts were seeded at a density of 5000/well onto 96-well plates and cultured overnight at 37° C. Then, cells were incubated with either 5% FBS or TGFβ (10 ng/mL, PeproTech) and/or rhNRG-1 (50 ng/mL, PeproTech). Cell-growth was measured by adding MTT to each well and by incubating for 4 hours at 37° C. The absorbance was recorded at 570 nm using an Epoch (BioTek) microplate reader.

RNA Extraction and Real-Time PCR

### Experiment A

The injected sections of the skin and the right lung were snap-frozen in liquid nitrogen at sacrifice. Skin and lung tissues were then homogenized using a Polytron homogenizer (Pt 2100; Kinematica, Littau, Switzerland) and total RNA was obtained by the GenElute Mammalian Total RNA Miniprep Kit (Sigma Aldrich).

Total RNA of lung and skin fibroblasts was extracted via the Absolutely Microprep RNA kit (Agilent).

Total RNA was transcribed to cDNA using random hexamers (TaqMan Reverse Transcription Reagents, Applied Biosystems).

Using TaqMan real-time PCR (Life Technologies), collagen type I (Mm00801666_g1), collagen type III (Mm01254476_m1) and Fibroblast specific protein-1 (Mm01210125_m1, s100a4) mRNA expression was analyzed in lung and skin tissue. In fibroblasts, collagen synthesis was determined by collagen type I (Rn01463848_m1) and type III (Rn01437681_m1).

### Experiment B

mRNA expression was analyzed by quantitative PCR using Taqman real-time PCR (Invitrogen) performed on the 7300 Real-Time PCR system (Applied Biosystems). Total RNA was extracted from cells or tissue with Mammalian Total RNA Miniprep kit (Qiagen). Following Taqman primers were used (Invitrogen); NRG-1 (Mm01212130_m1), Procollagen1a1 (COL1A1, Mm00801666_g1), procollagen3a1 (COL3A1, Mm01254476_m1), fibronectin-1 (Mm01256744_m1), fibroblast specific protein-1 (FSP-1 or s100a4, Mm00803372_g1), matrix metallopeptidase-2 (MMP2, Mm00439498_m1), matrix metallopeptidase-9 (MMP9, Mm00442991_m1), metalloprotease inhibitor-1 (TIMP1, Mm00441818_m1), endothelin-1 (Mm00438656_m1), interleukin-6 (IL6, Mm00446190_m1), interleukin-1β (IL1β, Mm00434228_m1), tumor necrosis factor-α (TNFα, Mm00443258_m1), Interferon-γ (IFNγ, Mm01168134), Transforming growth factor-β1 (TGF-β1, Mm01178820_m1) and inducible nitric oxide (iNOS, Mm00440502_m1).

Microarray

Fibroblasts were obtained from 8 different C57BL/6 mice. Primary fibroblasts were treated with either PBS (n=4) or rhNRG-1 (n=4; 50 ng/mL, PreproTech). RNA was extracted by micro-RNA Isolation kit (Sigma-Aldrich). RNA quantity was determined by NanoDrop 2000 (Thermo scientific) and integrity by Experion Bioanalyzer (Bio-Rad). For each experiment, 100 ng RNA was labeled using Illumina® TotalPrep RNA Amplification Kit (Ambion) and then hybridized on the Illumina MouseRef-8 v2.0 Expression BeadChip. Scanning was performed on a Bead Array Reader (I-Scan; Illumina). Analysis was performed using R 3.2.2 Statistical Software. Network and Pathway analysis was performed using the Ingenuity Pathway Analysis (IPA) software.

Data Analysis and Statistics

### Experiment A

Data are expressed as means±SEM. Differences between groups were analyzed by one-way ANOVA with Bonferroni post-hoc test. Western blots were subjected to densitometric analysis using ImageJ 1.42 software. Statistical significance was defined as P<0.05. All statistical analyses were done using Graph Pad Prism 5 and IBM SPSS Statistics 22 software.

### Experiment B

Data are expressed as means±SEM. Differences between groups were analyzed by one-way or two-way ANOVA with Bonferroni corrections for multiple comparisons. Western blots were subjected to densitometric analysis using ImageJ 1.42 software. Mortality was analyzed using the Gehan-Breslow-Wilcoxon test. Statistical significance was defined as P<0.05. All statistical analyses were done using GraphPad Prism 5 and IBM SPSS Statistics 22.

II. Results

### Example 1: Attenuation of Bleomycin-Induced Dermal Fibrosis

### Experiment 1A

In order to study the anti-fibrotic effect of NRG-1 on bleomycin-induced dermal fibrosis, changes were measured in bleomycin-treated mice with and without NRG-1 administration.

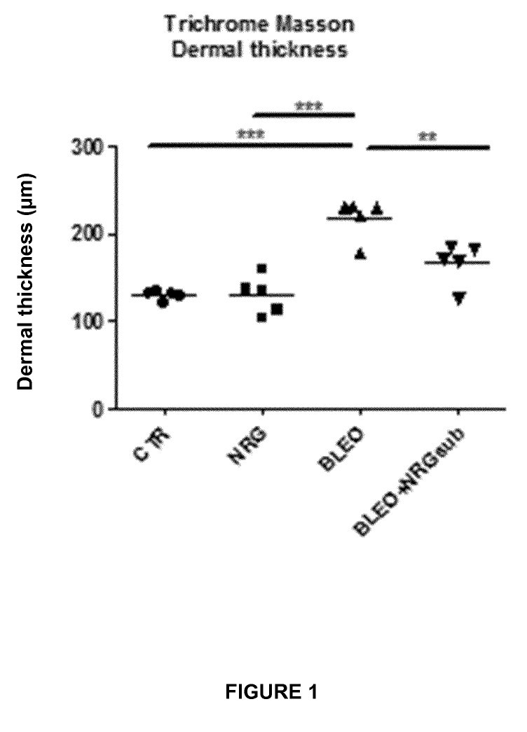

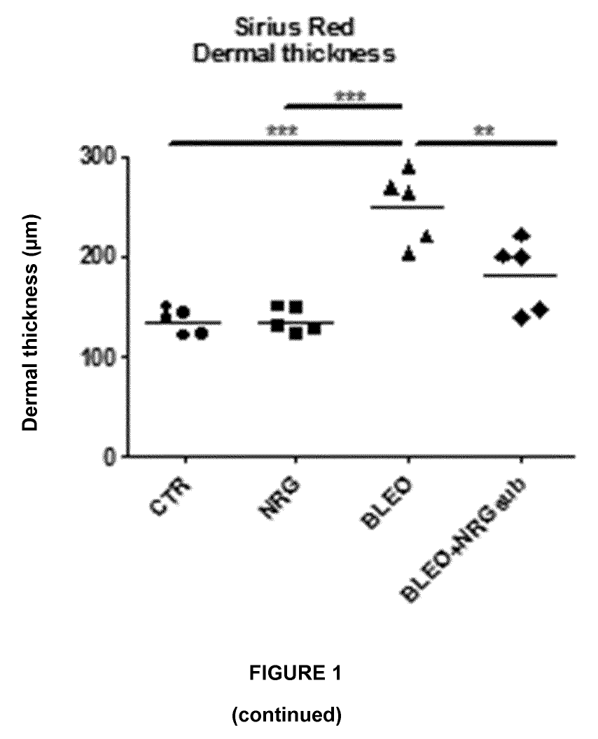

Skin sections were stained with Sirius red and Masson's trichome to determine the deposition of collagen and the dermal thickness. Bleomycin-treated mice showed an increased dermal thickness when compared with the PBS-treated mice and the collagen matrix partially replaced the subcutaneous layer of fat in the hypodermis. Mice treated with both bleomycin and NRG-1 showed a significant decrease of dermal thickness compared with the bleomycin-treated mice, which points to less collagen accumulation in the dermis (FIG. 1).

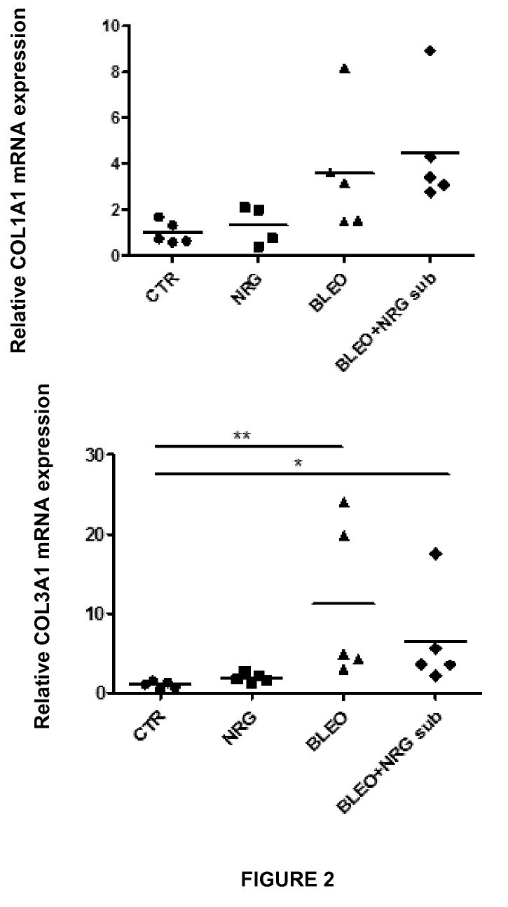

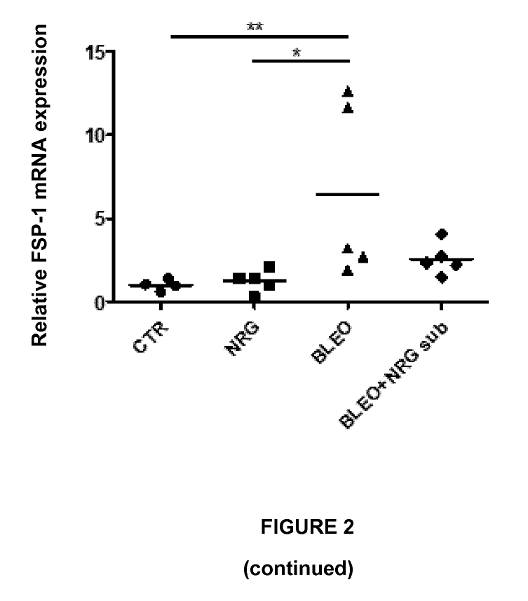

mRNA expression of collagen type I (COL1A1), collagen type III (COL3A1), and fibroblast specific protein-1 (FSP-1) was determined in the skin tissue. Collagen type I is the major component of extracellular matrix in skin, but also collagen type III and fibroblast specific protein-1 (FSP-1) are interesting markers for skin fibrosis. There was no significant difference in relative COL1A1 mRNA expression between groups. COL3A1 and FSP-1 were significantly upregulated in the bleomycin-treated mice in comparison with control mice. NRG-1 decreases COL3A1 and FSP-1 mRNA expression (FIG. 2).

It is observed that the neuregulin protein is capable of significantly reducing dermal fibrosis induced by subcutaneous injection of bleomycin.

### Experiment 1B

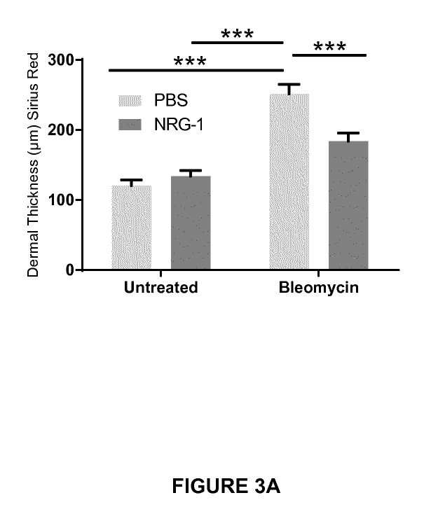

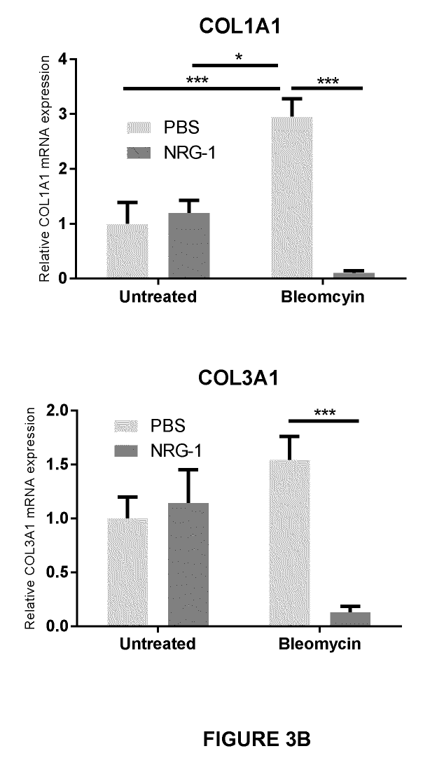

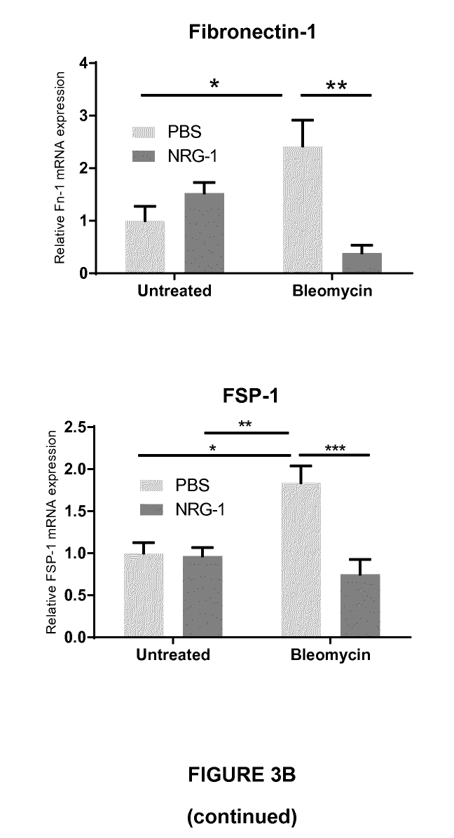

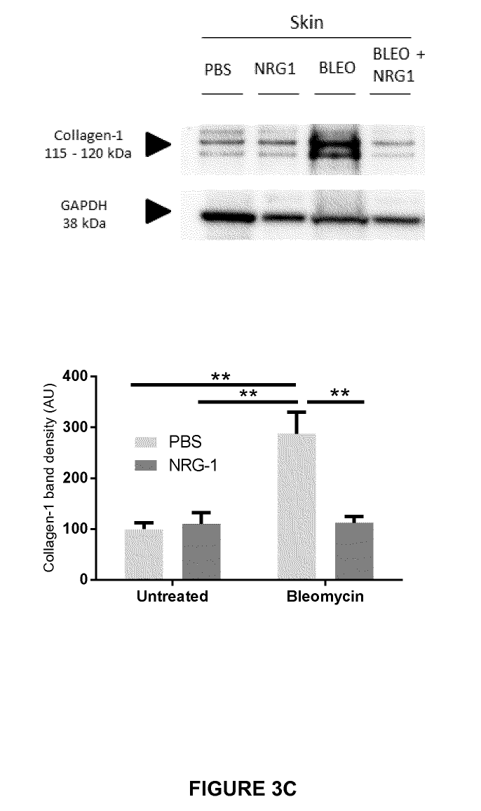

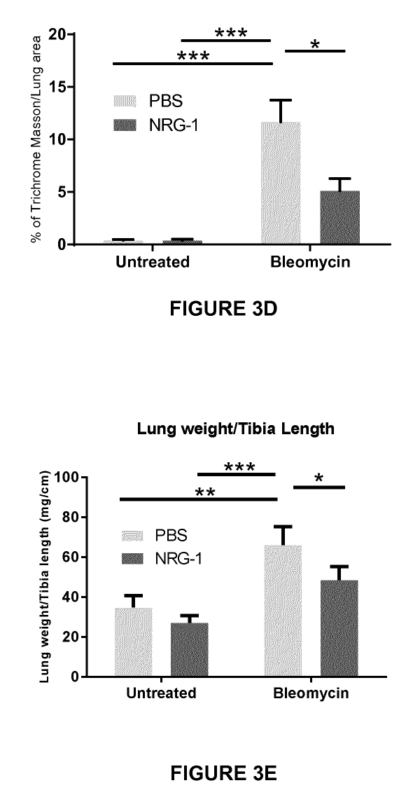

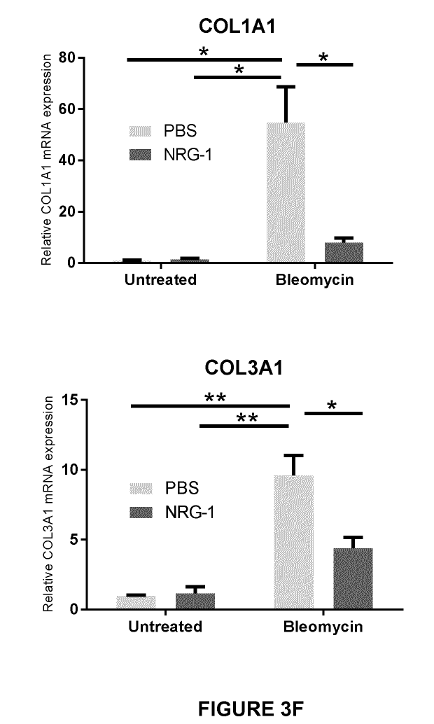

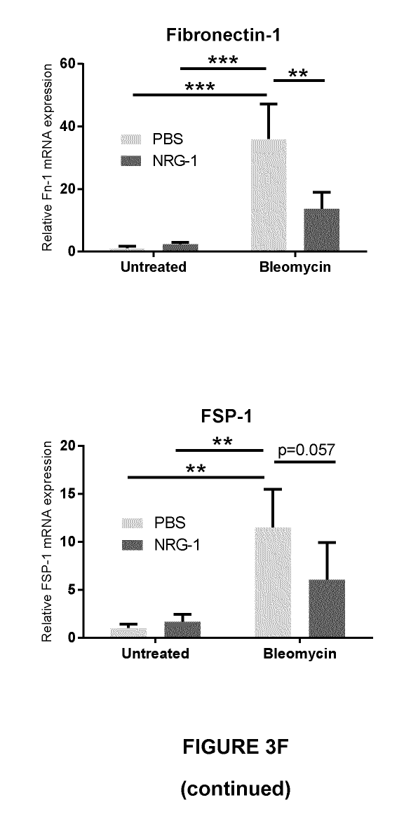

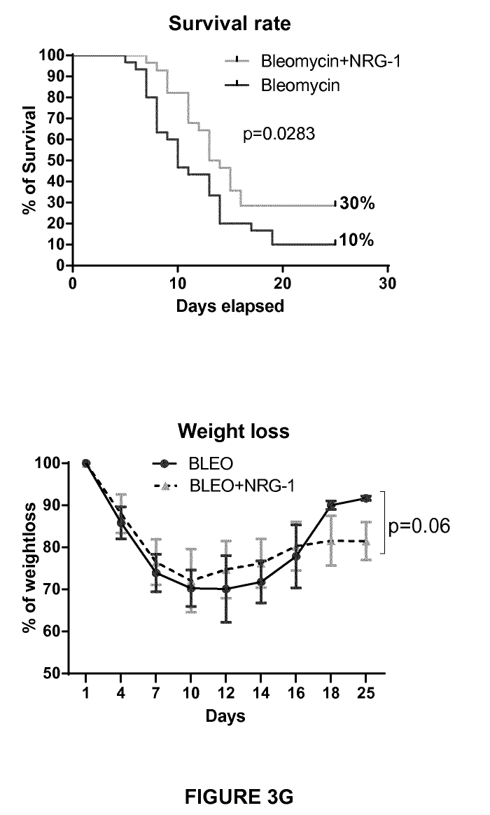

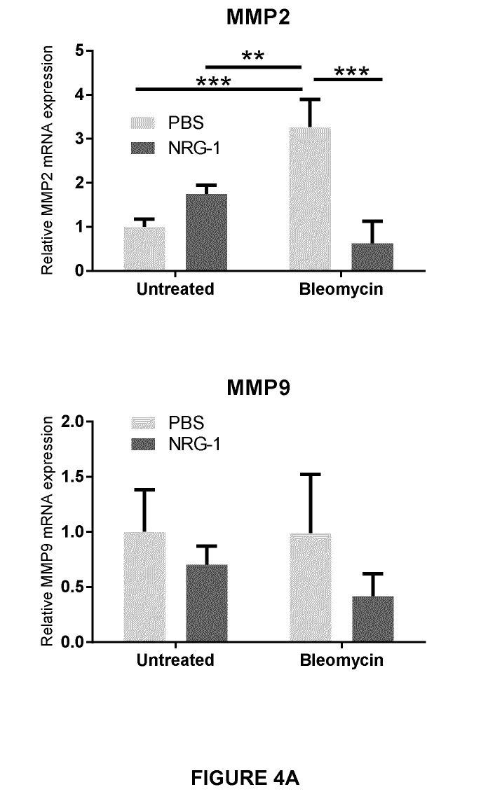

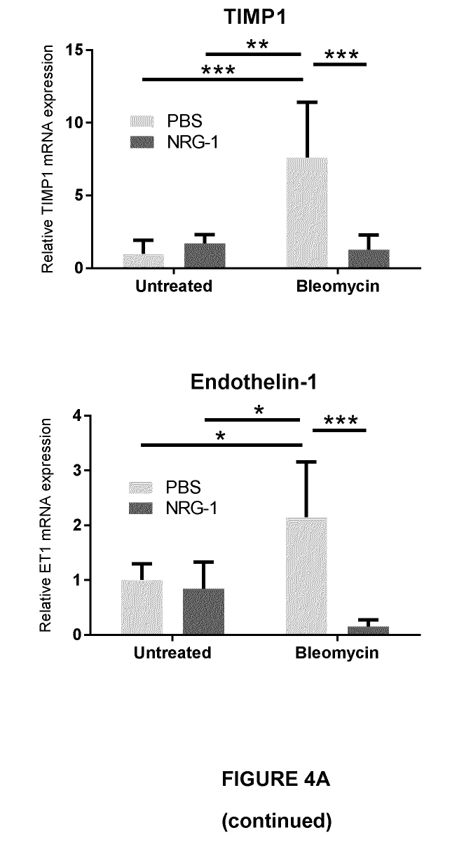

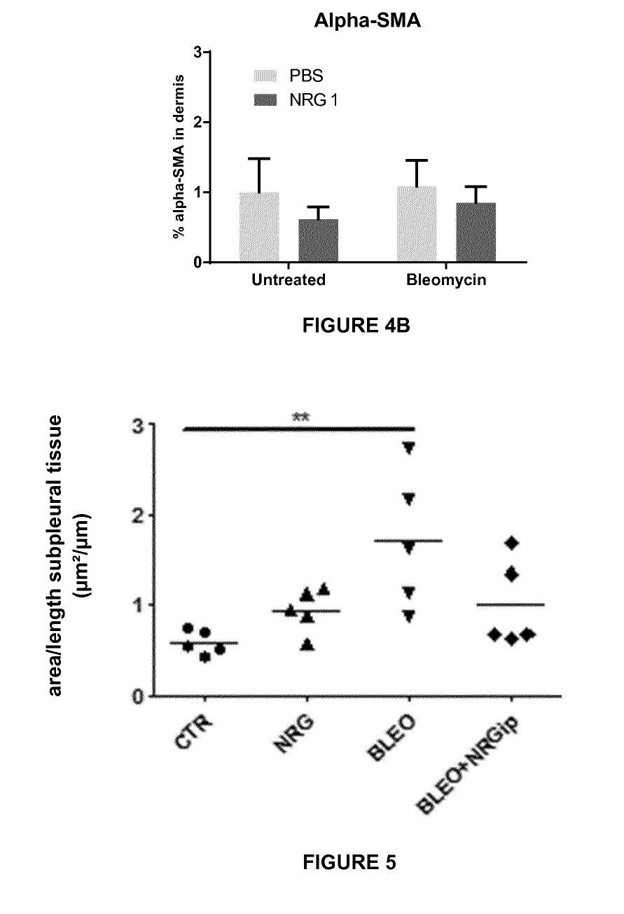

Upon Masson's trichome staining it was observed that subcutaneous bleomycin injections induced dermal fibrosis characterized by accumulation of ECM in the dermis. NRG-1 significantly attenuated dermal thickness with 65 μm, which indicates there was less ECM accumulation (FIG. 3A). Consistently NRG-1 prevented the accumulation of collagen-1 and the upregulation of COL1A1, COL3A1, fibronectin-1 and FSP-1 mRNA expression in skin tissue (FIG. 3B-C). MMP's, TIMP's and endothelin-1 are upregulated in most forms of fibrosis, and upregulation of these markers was also prevented by NRG-1 treatment (FIG. 4A). Because fibroblasts can convert to myofibroblasts in some forms of fibrosis, αSMA staining was performed, but bleomycin did not induce myoblast formation in our studies (FIG. 4B).

### Example 2: Attenuation of Bleomycin-Induced Pulmonary Fibrosis

### Experiment 2A

In order to study the anti-fibrotic effect of NRG-1 on bleomycin-induced pulmonary fibrosis, changes were measured in bleomycin-treated mice with and without NRG-1 administration.

Sections of the left lung were stained with Sirius red and analyzed under cross-polarized light to determine the fibrosis deposition. Lung fibrosis was analyzed by measuring the area (μm²) of fibrosis normalized to the length of the visceral pleura (μm). Histological examination showed subpleural fibrotic lesions in the bleomycin-treated mice. Mice treated with both NRG-1 and bleomycin showed less fibrotic deposition per subpleural tissue length (FIG. 5).

Another interesting parameter to assess lung fibrosis was lung weight. Lung weight normalized by tibia length was significantly upregulated in the bleomycin-treated group (FIG. 6). Mice that were given both NRG-1 and bleomycin, showed a significantly lower lung weight in comparison with the bleomycin-treated group.

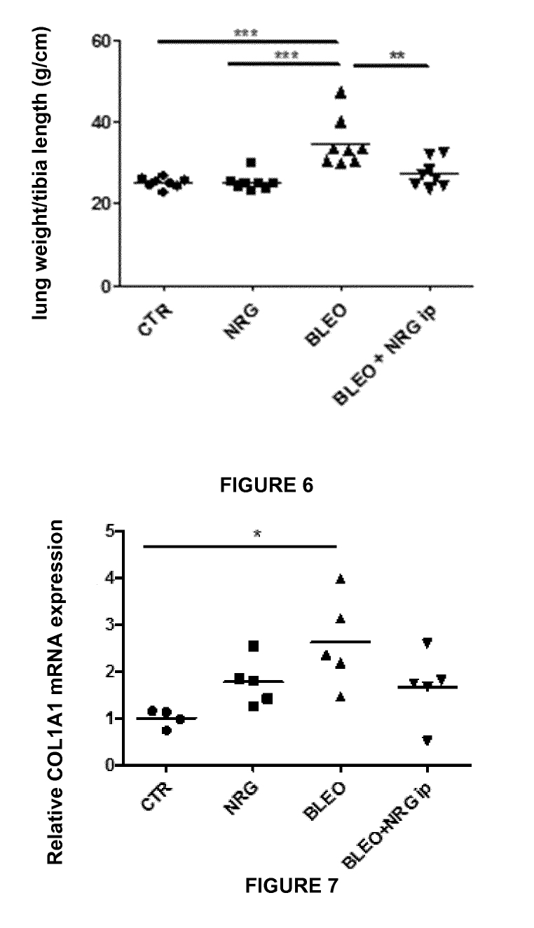

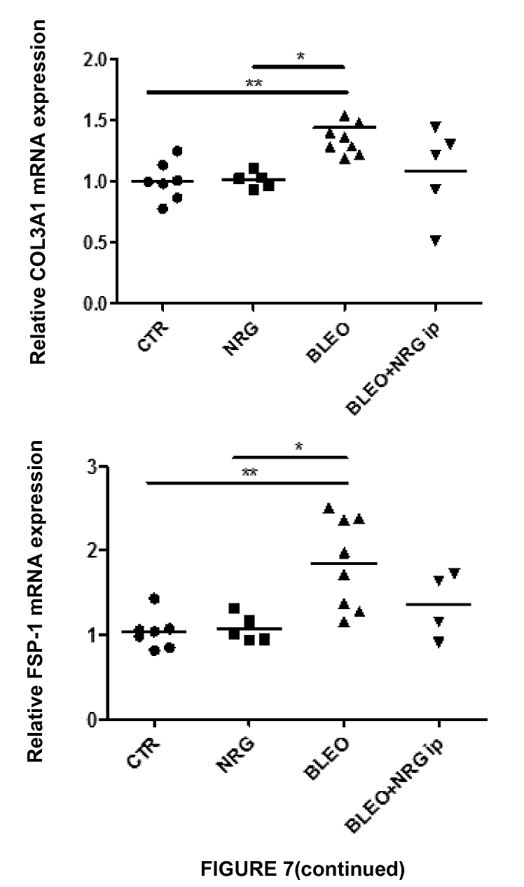

mRNA expression of collagen type I (COL1A1), collagen type III (COL3A1), and fibroblast specific protein-1 (FSP-1) was determined in the lung tissue. Collagen type I and type III are the most important fibrotic markers upregulated in pulmonary fibrosis. Another interesting marker is the fibroblast specific protein-1 (FSP-1) (Lawson et al. 2005). Relative COL1A1, COL3A1 and FSP-1 mRNA expression were significantly upregulated in the bleomycin-treated mice in comparison with control mice (FIG. 7). NRG-1 decreased expression of these fibrotic markers in fibrotic lungs.

It is observed that the neuregulin protein is capable of significantly reducing pulmonary fibrosis induced by subcutaneous injection of bleomycin.

### Experiment 2B

We tested the effects of NRG-1 in a model of pulmonary fibrosis, a disease with high mortality. It was observed that a single intratracheal injection induced pulmonary fibrosis (12%; P<0.001; FIG. 3D). Animals treated with NRG-1 showed significantly less pulmonary fibrosis (5%; P<0.001; FIG. 3D) and had decreased lung weights (FIG. 3E). Furthermore, mice treated with NRG-1 showed less upregulation of COL1A1, COL3A1, fibronectin-1 and FSP-1 mRNA in lung tissue (FIG. 3F). Furthermore, NRG-1 increased survival in this model from 10% to 29% (P<0.05; FIG. 3G).

### Example 3: Effect of NRG-1 on Lung and Skin Fibroblasts and their Collagen Synthesis

### Example 3A

To study the effect of NRG-1 on fibroblasts and there collagen synthesis, lung and skin fibroblasts were cultured and exposed to NRG-1.

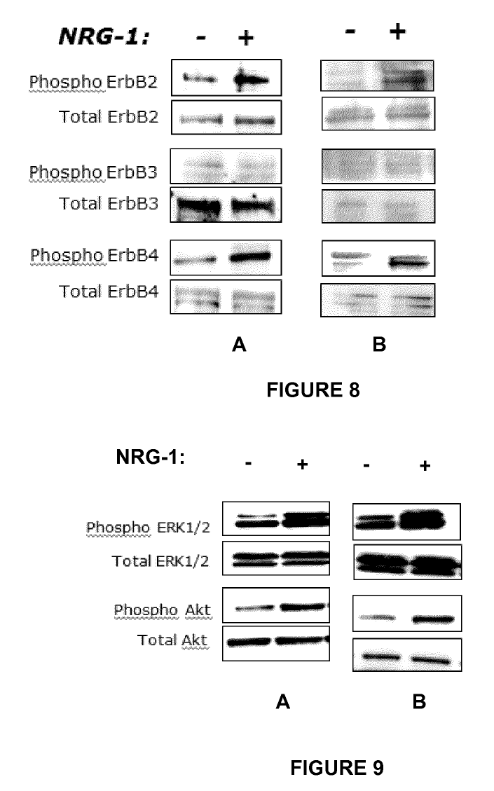

Both skin and lung fibroblasts expressed ErbB2, 3 and 4 receptors and exposure to NRG-1 led to rapid phosphorylation of ErbB2 and 4 (FIG. 8).

Akt and ERK1/2 pathways function downstream of neuregulin. Treatment of skin and lung fibroblasts with NRG-1 led to phosphorylation of Akt and ERK1/2, and thus to activation of the respective pathways (FIG. 9).

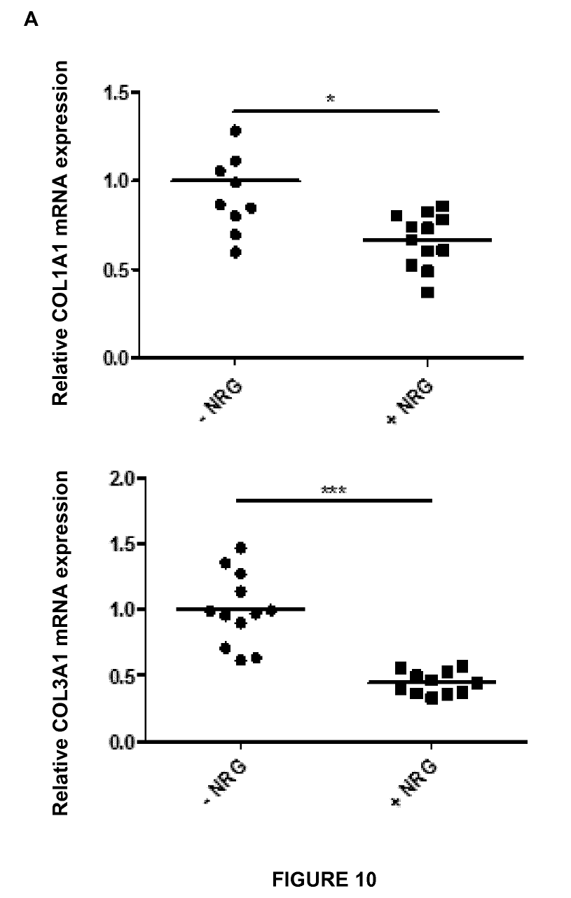

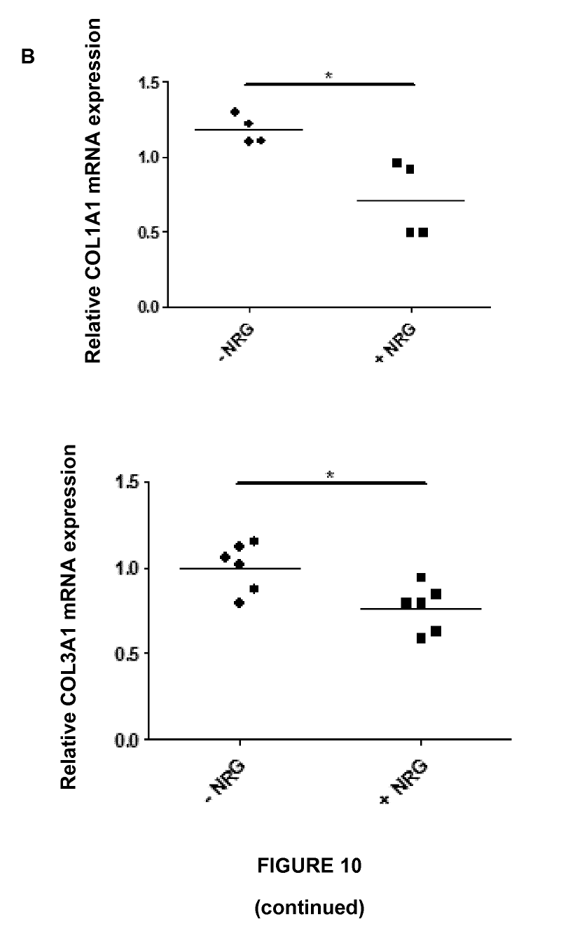

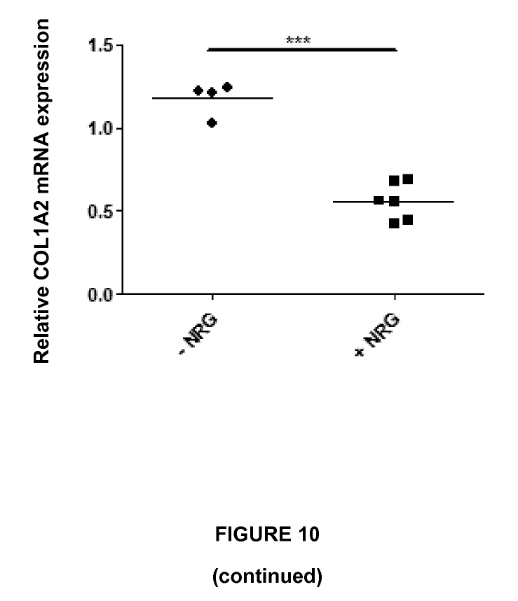

FIG. 10 shows that treatment of fibroblasts with NRG-1 significantly downregulated expression of collagen type I and type III.

In vitro, it has been shown here that NRG-1 activates the ErbB2 and ErbB4 receptors present in skin and lung fibroblasts, and the downstream Akt and ERK1/2 pathways. Furthermore, collagen type I and type III synthesis in skin and lung fibroblasts is attenuated by NRG-1. These findings support a role for NRG-1 as a therapeutic agent for the prevention, treatment and/or delay of dermal and fibrotic pulmonary disorders.

### Example 3B

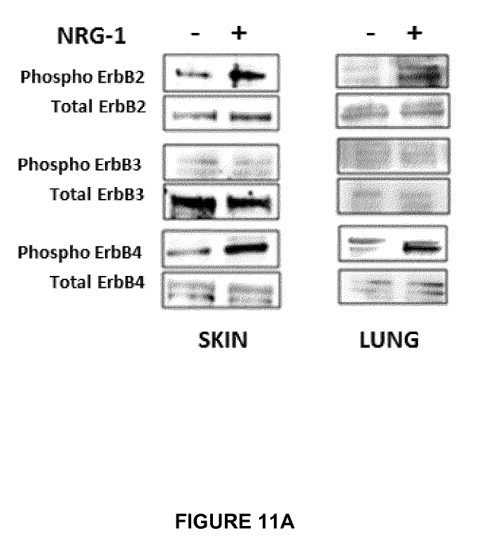

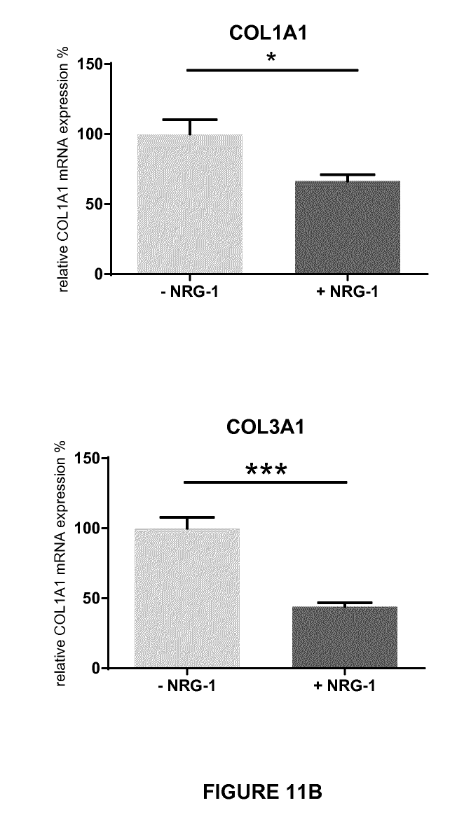

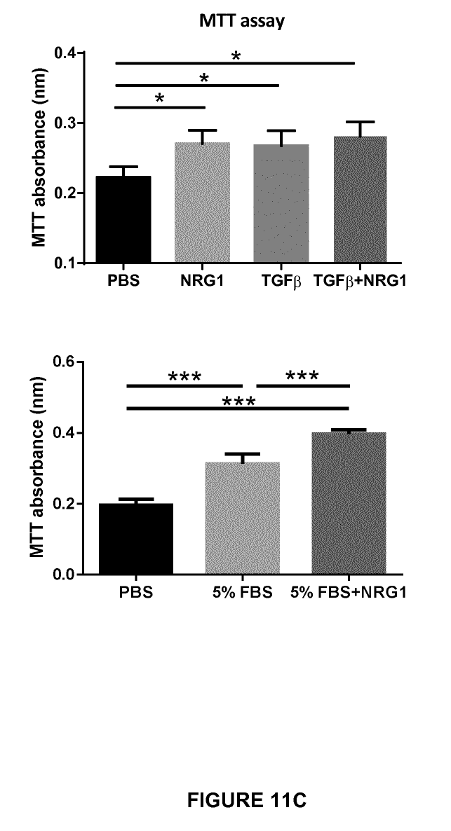

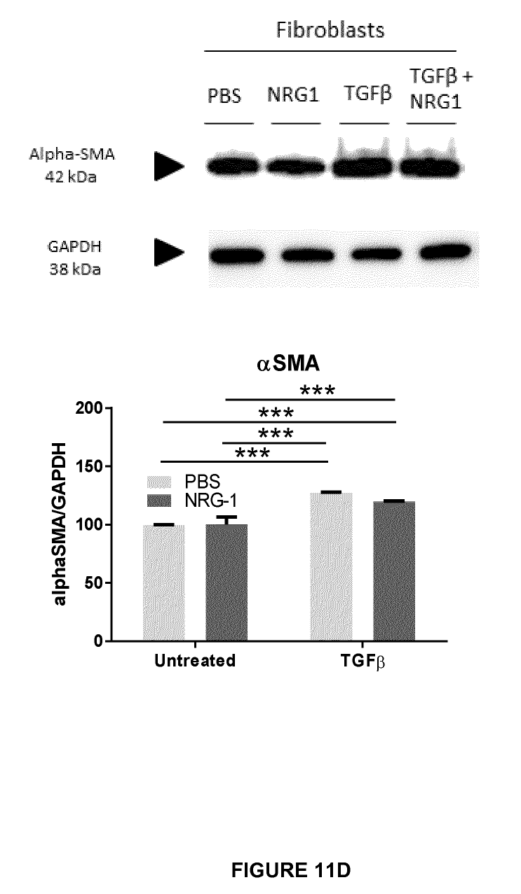

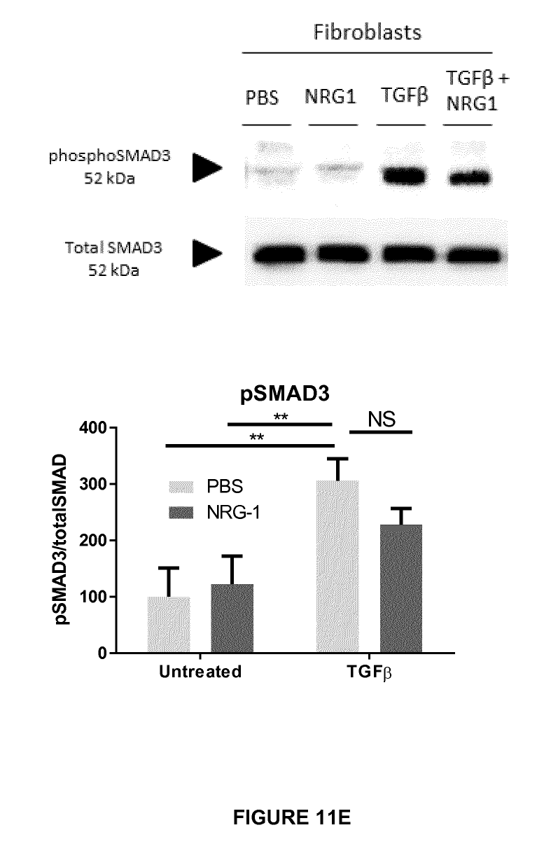

To test whether NRG-1 has direct effects on fibroblasts, we studied the effects of NRG-1 on primary cultured fibroblasts. FIG. 11A shows expression of ErbB2, ErbB3 and ErbB4 receptors in fibroblasts isolated from skin and lung tissue. Treatment of fibroblasts with NRG-1 attenuated stress-induced upregulation of COL1A1 and COL3A1 mRNA, implying a direct inhibitory effect of NRG-1 on fibroblasts (FIG. 11B). An MTT assay showed that NRG-1 had no anti-proliferative effect. In contrast, it induced proliferation of fibroblasts (FIG. 110). Furthermore, NRG-1 does not play a role in fibroblast-to-myofibroblast differentiation as NRG-1 had no effect on TGFβ-induced upregulation of αSMA (FIG. 11D). Moreover, NRG-1 did not significantly inhibit TGFβ-induced upregulation of phosphoSMAD3, suggesting that NRG-1 does not influence the TGFβ/SMAD signaling pathway (FIG. 11E).

### Example: Prevention and Treatment of Liver Fibrosis

The effect of neuregulin on pathological liver fibrosis is demonstrated in a mouse model of Nonalcoholic Steatohepatitis (NASH). Briefly C57BL/6 adult mice are used in a common dietary animal model with the methionine-choline deficient (MCD) diet. Animals are fed with this diet for 6 or 10 weeks in order to develop NASH with early fibrosis in time dependent manner. Animals are randomized to (1) treatment with rhNRG-1β intraperitoneal injection (20 μg·kg⁻¹·day⁻¹, PeProtech), (2) treatment with rhNRG-1β subcutaneous injection, or (3) no treatment. Control mice were treated with either (1) rhNRG-1β or (2) vehicle (PBS). The development of liver fibrosis is compared in the 2 groups at different time points as described above.

To study fibrosis regardless of the disease type, 0014 animal model of fibrosis is used on C57BL/6 mice. Briefly, a 0014 solution is administered intraperitoneally three times a week during 4 weeks (0.021 mole/kg, 20 μL, body weight). Animals are then randomized to either control or treatment with NRG-1 as indicated above and the development of liver fibrosis is compared in the 2 groups at different time points.

Liver samples are taken for analysis from all experimental groups at different time points. To validate the development and stage of fibrosis, histopathology analysis is performed with trichrome and Sirius red staining for collagen and connective tissue as described above.

Fibroblast specific ErbB4 KO mice are used with the same experimental protocols for developing NASH and liver fibrosis and treatment and non-treatment groups are compared.

It is observed that NRG-1 is capable of preventing and treating liver fibrosis.

### Example 5: NRG-1 Downregulates ECM-Related and Inflammatory Genes

To identify pathways involved in the anti-fibrotic effects of NRG-1, we performed microarray analysis on primary fibroblasts of 8 separate mice treated with either PBS (n=4) or NRG-1 (n=4). We identified a total of 960 differentially expressed genes (DEGs); 450 were down-regulated whereas 510 were up-regulated by NRG-1 treatment. Hierarchical clustering of the obtained DEGs shows a clear distinction of the samples by their corresponding treatment group. NRG-1-treated fibroblasts showed a significant decrease in expression of several ECM-related genes, summarized in Table 1.

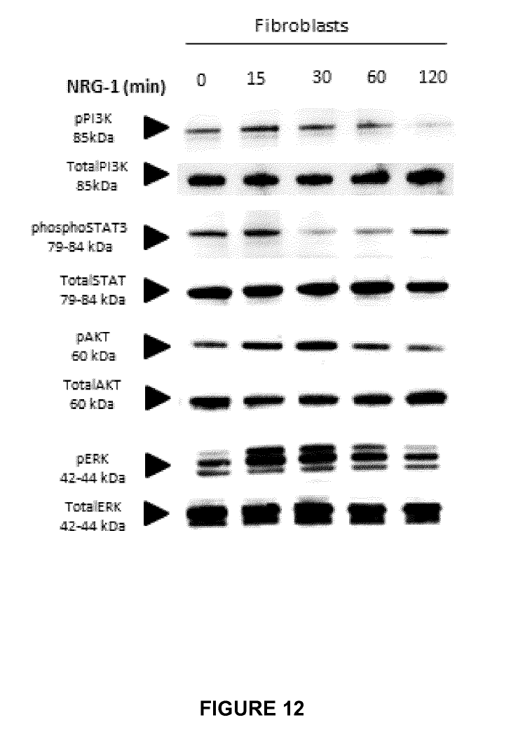

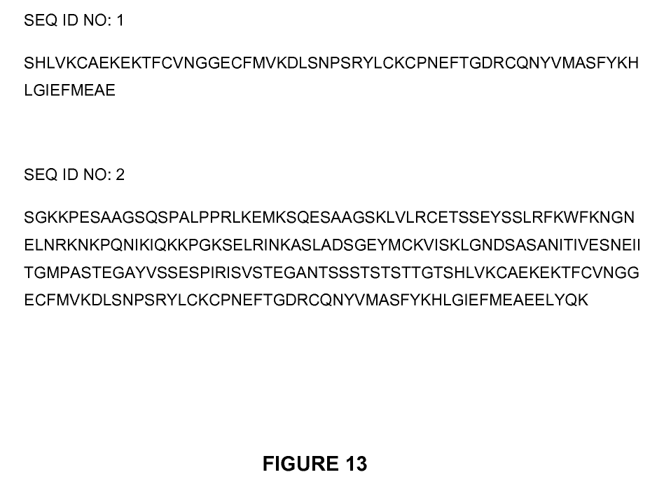

Ingenuity Pathway Analysis (IPA) connected ECM-related genes in a fibroblast-specific network illustrated in FIG. 15A. IPA also determined over-expressed signaling pathways and divided data into disease and biological functions. In Table 2, top 10 significantly enriched signaling pathways are shown. Western blot analysis was performed on cultured fibroblasts exposed to NRG-1. Consistent with the microarray results, Akt and ERK1/2 were activated, while PI3K and STAT3 were downregulated (FIG. 12). The majority of the other significantly overrepresented canonical pathways were involved in inflammation. Disease associations are summarized in Table 3 and confirmed that connective tissue development is inhibited. It also suggested that NRG-1 has anti-inflammatory effects. Finally, we performed an Upstream Regulator Analysis in IPA to identify the cascade of upstream transcriptional regulators that can explain the observed gene expression changes (Table 4). Inflammatory cytokines were predicted to be the most inhibited upstream regulators, but also effects of chemical agents bleomycin an lipopolysaccharide were inhibited.

Thus, IPA revealed several genes involved in fibrogenic activities that were downregulated by NRG-1 treatment. Furthermore, JAK/STAT signaling played an important role in the NRG-1 mechanisms.

TABLE 1
Significantly decreased extracellular
matrix glycoproteins and proteoglycans

| | Fold | |
|---|---|---|
| Gene | Change | Protein Name |
| Itgav | 10.32 | integrin alpha V |
| P4ha2 | 8.5 | procollagen-proline, 2-oxoglutarate |
| | | 4-dioxygenase (proline 4-hydroxylase), |
| | | alpha II polypeptide |
| Edil3 | 6.93 | EGF-like repeats and discoidin I-like |
| | | domains 3 |
| P4ha1 | 6.26 | procollagen-proline, 2-oxoglutarate |
| | | 4-dioxygenase (proline 4-hydroxylase), |
| | | alpha 1 polypeptide |
| Sparc | 6.08 | secreted acidic cysteine rich glycoprotein |
| Fbln2 | 4.86 | fibulin 2 |
| Thbs1 | 4.86 | thrombospondin 1 |
| Adipoq | 4.81 | adiponectin, C1Q and collagen domain containing |
| Nell2 | 4.8 | NEL-like 2 |
| Ltbp1 | 4.76 | latent transforming growth factor beta binding |
| | | protein 1 |
| Mmp23 | 4.66 | matrix metallopeptidase 23 |
| Nyx | 4.52 | nyctalopin |
| Col28a1 | 4.18 | collagen, type XXVIII, alpha 1 |
| Col11a1 | 4.16 | collagen, type XI, alpha 1 |
| Vwde | 4.16 | von Willebrand factor D and EGF domains |
| Pxdn | 4.09 | peroxidasin homolog (Drosophila) |
| Fgl1 | 3.48 | fibrinogen-like protein 1 |
| Mmp27 | 3.21 | matrix metallopeptidase 27 |
| Wisp1 | 3.17 | WNT1 inducible signaling pathway protein 1 |
| Fbln1 | 3.16 | fibulin 1 |
| Ntng1 | 3.05 | netrin G1 |
| Omd | 2.92 | osteomodulin |
| Fgl2 | 2.85 | fibrinogen-like protein 2 |
| Podn | 2.83 | podocan |
| Col6a1 | 3.8 | collagen, type VI, alpha 1 |
| Timp3 | 2.8 | tissue inhibitor of metalloproteinase 3 |
| Mmp1a | 2.72 | matrix metallopeptidase 1a (interstitial collagenase) |
| Impg1 | 2.61 | interphotoreceptor matrix proteoglycan 1 |
| Tnc | 2.6 | tenascin C |
| Thbs3 | 2.29 | thrombospondin 3 |
| Col24a1 | 2.08 | collagen, type XXIV, alpha 1 |
| Mmp14 | 2.01 | matrix metallopeptidase 14 (membrane-inserted) |
| Efemp2 | 2 | epidermal growth factor-containing fibulin-like |
| | | extracellular matrix protein 2 |
| Matn4 | 2 | matrilin 4 |
| Ogn | 2 | osteoglycin |

TABLE 2
Canonical pathways significantly associated with NRG-1 treatment

| | | | |
|---|---|---|---|
| Ingenuity | | | |
| Canonical | | | |
| Pathways | -log(p-value) | Ratio | Genes |

Top 10 Signaling pathways

| | | | |
|---|---|---|---|
| JAK/Stat Signaling | 3.26E00 | 1.39E−01 | STAT3, STAT4, PIK3R2, STAT1, STAT2, |
| | | | STAT6, CISH, SOCS2, AKT2, MAPK3 |
| CXCR4 Signaling | 2.51E00 | 9.21E−02 | PRKCE, CXCL12, ITPR2, ELM02, PXN, RHOJ, |
| | | | AKT2, PIK3R2, RHOT1, GNG13, RHOV, ELMO1, |
| | | | RND3, MAPK3 |
| GM-CSF Signaling | 2.51E00 | 1.29E−01 | STAT3, PIK3R2, BCL2A1, STAT1, CISH, PPP3CA, |
| | | | AKT2, MAPK3 |
| Activation of IRF by | 2.42E00 | 1.25E−01 | TANK, DHX58, IRF9, STAT1, STAT2, ISG15, DD |
| Cytosolic Pattern | | | X58, ZBP1 |
| Recognition Receptors | | | |
| Production of Nitric | 2.24E00 | 8.33E−02 | PRKCE, MAP2K7, TNFRSF1A, MAP3K14, PPM1L, |
| Oxide and Reactive | | | RHOJ, AKT2, PIK3R2, STAT1, PPP1CB, RHOT1, |
| Oxygen Species in | | | RHOV, RND3, PPP1R11, MAPK3 |
| Macrophages | | | |
| Gαq Signaling | 2.22E00 | 8.84E−02 | PRKCE, ITPR2, ADRA1B, PPP3CA, RHOJ, AKT2, |
| | | | GYS2, PIK3R2, RHOT1, GNG13, RHOV, RND3, MAPK3 |
| Nitric Oxide Signaling | 2.19E00 | 1E−01 | PRKCE, ITPR2, PIK3R2, HSP90B1, ADRB1, GUCY2D, |
| in the Cardiovascular | | | ATP2A2, HSP90AB1, AKT2, MAPK3 |
| System | | | |
| EGF Signaling | 2.18E00 | 1.25E−01 | MAP2K7, ITPR2, STAT3, PIK3R2, STAT1, AKT2, |
| | | | MAPK3 |
| Regulation of the | 2.16E00 | 8.15E−02 | MAP2K7, CDH2, PARD6A, CTNNB1, ZEB2, TCF7L1, |
| Epithelial- | | | AKT2, WNT2B, NCSTN, STAT3, PIK3R2, MAML1, |
| Mesenchymal | | | ARAF, FGF9, MAPK3 |
| Transition Pathway | | | |
| IL-3 Signaling | 2.15E00 | 1.13E−01 | PRKCE, STAT3, PIK3R2, STAT1, STAT6, PPP3CA, |
| | | | AKT2, MAPK3 |

TABLE 3
Disease and biological functions significantly associated with NRG-1 treatment

| | | | |
|---|---|---|---|
| | Diseases of Functions | | |
| Categories | Annotation | p-Value | z-score |

Activated

| | | | |
|---|---|---|---|
| Cell Death and Survival | cell death of tumor cell lines | 3.00E−04 | 2.982 |
| Cardiovascular Disease | myocardial infarction | 7.81E−03 | 1.89 |
| Nervous System Development and | formation of neurons | 5.01E−03 | 0.883 |
| Function | | | |

Inhibited

| | | | |
|---|---|---|---|
| Dermatological Diseases and | damage of skin | 1.02E−03 | −0.714 |
| Conditions | | | |
| Hematological System | quantity of megakaryocytes | 1.28E−02 | −1.172 |
| Development and Function | | | |
| Protein Synthesis | quantity of cytokine | 8.51E−03 | −1.264 |
| Hematological System | quantity of lymphocytes | 5.92E−03 | −1.296 |
| Development and Function | | | |
| Cell-mediated Immune Response | differentiation of CD4 + T- | 2.25E−03 | −1.387 |
| | lymphocytes | | |
| Inflammatory Response | inflammation of body region | 1.11E−02 | −1.438 |
| Connective Tissue Development | quantity of connective tissue | 8.22E−03 | −1.608 |
| and Function, Tissue Morphology | | | |
| Hematological System | quantity of neutrophils | 2.34E−03 | −1.678 |
| Development and Function | | | |
| Cellular Function and Maintenance, | function of connective tissue | 8.98E−03 | −2 |
| Tissue Development | cells | | |
| Cancer, Organismal Injury and | growth of malignant tumor | 1.18E−02 | −2.617 |
| Abnormalities | | | |
| DNA Replication, Recombination, | DNA damage | 1.35E−02 | −2.689 |
| and Repair | | | |
| Hematological System | quantity of phagocytes | 6.84E−03 | −3.102 |
| Development and Function, | | | |

TABLE 4
Activated and inhibited upstream regulators

| | | Predicted | | |
|---|---|---|---|---|
| Upstream | | Activation | Activation | p-value of |
| Regulator | Molecule Type | State | z-score | overlap |
| SOCS1 | other | Activated | 3.118 | 5.93E−04 |
| PPARG | ligand-dependent | Activated | 2.960 | 2.24E−02 |
| | nuclear receptor | | | |
| IFNA4 | cytokine | Inhibited | −2.164 | 1.75E−03 |
| TNF | cytokine | Inhibited | −2.171 | 6.12E−03 |
| IL12A | cytokine | Inhibited | −2.190 | 5.18E−02 |
| IL12B | cytokine | Inhibited | −2.190 | 5.18E−02 |
| bleomycin | chemical drug | Inhibited | −2.200 | 5.30E−01 |
| TLR3 | transmembrane | Inhibited | −2.206 | 2.02E−03 |
| | receptor | | | |
| IL3 | cytokine | Inhibited | −2.217 | 7.49E−02 |
| IL24 | cytokine | Inhibited | −2.236 | 1.34E−02 |
| MAPK8 | kinase | Inhibited | −2.236 | 1.00E00 |
| OSM | cytokine | Inhibited | −2.384 | 8.88E−02 |
| IL27 | cytokine | Inhibited | −2.388 | 1.92E−01 |
| EPO | cytokine | Inhibited | −2.390 | 1.85E−01 |
| GDF2 | growth factor | Inhibited | −2.400 | 1.05E−01 |
| IL6 | cytokine | Inhibited | −2.408 | 2.05E−02 |
| IL2 | cytokine | Inhibited | −2.417 | 4.15E−01 |
| IFN Beta | group | Inhibited | −2.498 | 4.74E−03 |
| STAT1 | transcription | Inhibited | −2.507 | 4.46E−06 |
| | regulator | | | |
| IL5 | cytokine | Inhibited | −2.519 | 2.22E−03 |
| CSF1 | cytokine | Inhibited | −2.537 | 1.07E−02 |
| IFN type 1 | group | Inhibited | −2.570 | 2.85E−04 |
| IFNA1/ | cytokine | Inhibited | −2.588 | 4.39E−03 |
| IFNA13 | | | | |
| IRF1 | transcription | Inhibited | −2.639 | 6.77E−04 |
| | regulator | | | |
| NFkB (complex) | complex | Inhibited | −2.783 | 1.61E−01 |
| Ifnar | group | Inhibited | −2.889 | 6.99E−05 |
| IFNA2 | cytokine | Inhibited | −2.905 | 7.87E−07 |
| Ifn | group | Inhibited | −2.939 | 1.37E−02 |
| IRF5 | transcription | Inhibited | −2.975 | 9.77E−04 |
| | regulator | | | |
| IFNG | cytokine | Inhibited | −3.078 | 2.52E−04 |
| lipopoly- | chemical drug | Inhibited | −3.088 | 2.99E−04 |
| saccharide | | | | |
| TLR4 | transmembrane | Inhibited | −3.372 | 2.86E−03 |
| | receptor | | | |
| IFNB1 | cytokine | Inhibited | −3.401 | 2.54E−05 |
| SP1 | transcription | Inhibited | −3.649 | 4.54E−03 |
| | regulator | | | |
| IRF7 | transcription | Inhibited | −3.684 | 5.67E−08 |
| | regulator | | | |
| Interferon | group | Inhibited | −3.761 | 1.90E−05 |
| alpha | | | | |
| IRF3 | transcription | Inhibited | −3.799 | 1.69E−04 |
| | regulator | | | |

### REFERENCES

Bersell K, Arab S, Haring B and Kuhn B. Neuregulin1/ErbB4 Signaling Induces Cardiomyocyte Proliferation and Repair of Heart Injury Cell 2009; 138:257-270.
Lindner D, Zietsch C, Moritz P. Differential expression of matrix metalloproteases in human fibroblasts with different origins. Biochemistry Research International. 2012
Wynn T A. Common and unique mechanisms regulate fibrosis in various fibroproliferative diseases. J Clin Invest. 2007 March; 117(3):524-9.
Wynn T A. Fibrotic disease and the T(H)1/T(H)2 paradigm. Nat Rev Immunol. 2004 August; 4(8):583-94.
Song Y1, Zhu L, Li M. Antifibrotic effects of crocetin in scleroderma fibroblasts and in bleomycin-induced sclerotic mice. Clinics. 2013 October; 68(10):1350-7.
Ichihara S, Senbonmatsu T, Price E, Jr, Angiotensin II type 2 receptor is essential for left ventricular hypertrophy and cardiac fibrosis in chronic angiotensin II-induced hypertension. Circulation 2001 104:346-351.
Olman M A, Mackman N, Gladson C L, Changes in procoagulant and fibrinolytic gene expression during bleomycin-induced lung injury in the mouse. J Clin Invest 1995 96:1621-1630.
Strutz F, Okada H, Lo C. Identification and characterization of a fibroblast marker: FSP1. J Cell Biol 1995 130:393-405.
Sanada S, Hakuno D, Higgins. IL-33 and ST2 comprise a critical biomechanically induced and cardioprotective signaling system. J Clin Invest 2007 117:1538-1549.
Takashima A. Establishment of Fibroblast Cultures. Current Protocols in Cell Biology. 2001 May.
Lemmens K, Doggen K, De Keulenaer G W. Activation of the neuregulin/ErbB system during physiological ventricular remodeling in pregnancy. Am J Physiol Heart Circ Physiol. 2011 March; 300(3):H931-42.
Santiago B1, Gutierrez-Cañas I, Dotor J, Palao G, Lasarte J J, Ruiz J, et al. Topical application of a peptide inhibitor of transforming growth factor-beta1 ameliorates bleomycin-induced skin fibrosis. J Invest Dermatol. 2005 September; 125(3):450-5.
Lawson W E1, Polosukhin V V, Zoia O, Stathopoulos G T, Han W, Plieth D, et al. Characterization of fibroblast-specific protein 1 in pulmonary fibrosis. Am J Respir Crit Care Med. 2005 Apr. 15; 171(8):899-907.
Raghu G1, Masta S, Meyers D, Narayanan A S. Collagen synthesis by normal and fibrotic human lung fibroblasts and the effect of transforming growth factor-beta. Am Rev Respir Dis. 1989 July; 140(1):95-100.

1-15. (canceled)

16. A method of treating, preventing and/or delaying a fibrotic skin disorder, a fibrotic lung disorder or liver cirrhosis in a mammal, said method comprising administering to said mammal a neuregulin (NRG) protein, thereby treating, preventing and/or delaying a fibrotic skin disorder, a fibrotic lung disorder or liver cirrhosis in a mammal.

17. The method of claim 16, wherein said fibrotic skin disorder is selected from the group consisting of sclerosis, preferably systemic sclerosis (or scleroderma), and hypertrophic scarring.

18. The method of claim 16, wherein said fibrotic lung disorder is idiopathic pulmonary fibrosis (IPF).

19. The method of claim 16, wherein said NRG protein reduces or prevents dermal fibrosis, pulmonary fibrosis, or liver fibrosis.

20. The method of claim 16, wherein said NRG protein suppresses collagen synthesis and/or fibroblast specific protein-1 (FSP-1) synthesis.

21. The method of claim 16, wherein said NRG protein activates the ERK1/2 and/or Akt signalling pathways.

22. The method of claim 16, wherein said NRG protein is a neuregulin-1 (NRG-1) protein, a neuregulin-2 (NRG-2) protein, a neuregulin-3 (NRG-3) protein, a neuregulin-4 (NRG-4) protein, or any mixture thereof, preferably a NRG-1 protein.

23. The method of claim 16, wherein said NRG protein is a type 1 NRG-1 protein.

24. The method of claim 16, wherein said NRG protein is the beta1 isoform of type 1 NRG-1 protein.

25. The method of claim 16, wherein said NRG protein comprises an EGF-like domain.

26. The method of claim 16, wherein said NRG protein is administered daily.

27. The method of claim 16, wherein said NRG protein is administered in a daily dose ranging from 0.01 to 100 μg/kg body weight.

28. The method of claim 16, wherein said mammal is a human.

29. The method of claim 16, wherein said NRG protein is administered in the form of a nucleic acid encoding said NRG protein.

30. The method of claim 16, wherein said NRG protein is provided in a pharmaceutical composition.

31. The method of claim 16, wherein said NRG protein decreases ECM-related genes.

32. The method of claim 16, wherein, wherein said NRG protein suppresses inflammation.
